(19) United States
(12) Patent Application Publication
(10) Pub. No.: US 2026/0250426 A1
(43) Pub. Date: Aug. 27, 2026

(54) AMPHIPHILIC CELLULOSE DERIVATIVES, METHODS OF MAKING, AND USES THEREOF

(71) Applicants: Virginia Tech Intellectual Properties, Inc., Blacksburg, VA (US); Purdue Research Foundation, West Lafayette, IN (US)

(72) Inventors: Kevin J. Edgar, Blacksburg, VA (US); Stella Petrova, Blacksburg, VA (US); Chengzhe Gao, West Lafayette, IN (US); Lynne Taylor, West Lafayette, IN (US)

(21) Appl. No.: 18/874,637

(22) PCT Filed: Jun. 13, 2023
(86) PCT No.: PCT/US2023/025216

§ 371 Date: Dec. 13, 2024

Related U.S. Application Data
(60) Provisional application No. 63/351,721, filed on Jun. 13, 2022.

(51) Int. Cl. C08B 3/12; A61K 9/16; A61K 9/20; A61K 31/4166; A61K 31/496; A61K 31/635

(52) U.S. Cl. CPC C08B 3/12; A61K 9/1652; A61K 9/2054; A61K 31/4166; A61K 31/496; A61K 31/635

(57) ABSTRACT

Described in several exemplary embodiments herein are cellulose derivatives and amorphous solid dispersions thereof that can contain one or more agents, optionally one or more poorly soluble agents.

### CROSS-REFERENCE TO RELATED APPLICATIONS

This application claims the benefit of and priority to co-pending U.S. Provisional Patent Application No. 63/351,721, filed on Jun. 13, 2022, entitled “AMPHIPHILIC CELLULOSE DERIVATIVES, METHODS OF MAKING, AND USES THEREOF,” the contents of which is incorporated by reference herein in its entirety.

### STATEMENT REGARDING FEDERALLY SPONSORED RESEARCH

This invention was made with government support under Grant No. PFI-RP 1827493 awarded by the National Science Foundation. The government has certain rights in the invention.

### TECHNICAL FIELD

The subject matter disclosed herein is generally directed to polymeric materials for drug delivery.

### BACKGROUND

The proportion of drugs designated as Biopharmaceutics Classification System (BCS) class II and IV compounds has increased in recent years and these poorly water soluble compounds now comprise a majority of the drugs in development. Oral delivery is a primary route of drug delivery. Compounds provided in an oral dosage form must first dissolve prior to absorption across the gastrointestinal epithelium. As such, there exists an urgent need for formulations and techniques to improve the solubility of compounds, particularly therapeutically relevant compounds.

Citation or identification of any document in this application is not an admission that such a document is available as prior art to the present invention.

### SUMMARY

Described in certain example embodiments herein are amphiphilic cellulose derivatives comprising a cellulose or a cellulose derivative, wherein one or more hydroxyl groups of the cellulose or the cellulose derivative are replaced by α,ω-dicarboxylic acid mono-ester (“ω-carboxyalkanoate ester”) substituents, optionally C₄ or higher ω-carboxyalkanoate ester substituents, any substituted derivatives thereof, or any combination thereof, and wherein the cellulose or the cellulose derivative is not crosslinked.

In certain example embodiments, the amphiphilic cellulose derivative does not comprise oligomeric anhydrides, poly(anhydrides), side chains thereof, or any combination thereof, optionally crosslinked or capable of forming crosslinks between hydroxyl groups of different molecules of the amphiphilic cellulose derivative.

In certain example embodiments, the ω-carboxyalkanoate ester substituents comprise a terminal carboxylic acid group or a monovalent salt thereof, optionally an alkali metal or a quaternary ammonium salt thereof.

In certain example embodiments, the ω-carboxyalkanoate ester substituents are linear C₄ or higher ω-carboxyalkanoate ester substituents chosen from succinate mono-ester substituents, glutarate mono-ester substituents, any substituted derivatives thereof, and any combination thereof, optionally wherein the substituted derivatives thereof comprise alkanoate substituents, alkyl substituents, hydroxyalkyl substituents, or any combination thereof.

In certain example embodiments, the amphiphilic cellulose derivative comprises a 1/1 or greater molar ratio of ω-carboxyalkanoate ester substituents to available hydroxyl groups, and/or wherein the amphiphilic cellulose derivative comprises an average of 25% or greater ω-carboxyalkanoate ester substituents based on total substituents.

In certain example embodiments, the cellulose derivative comprises an average degree of substitution of available hydroxyl groups of 1.8 or greater.

In certain example embodiments, the cellulose derivative comprises ester substituents having the formula —C(═O)R, ether substituents having the formula —OR, or any combination thereof, optionally wherein R is independently chosen at each occurrence from hydroxyl groups, alkyl ester substituents, alkoxide ester substituents, and any combination thereof.

In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the cellulose derivative comprises acetate substituents, propionate substituents, butyrate substituents, hydroxyalkyl substituents, hydroxy functional polyether substituents, or any combination thereof.

In certain example embodiments, the cellulose derivative is cellulose acetate, cellulose propionate, cellulose acetate propionate, cellulose acetate butyrate, hydroxypropyl cellulose, hydroxyethyl cellulose, or any combination thereof.

In certain example embodiments, the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxypropyl cellulose, hydroxyethyl cellulose, or any combination thereof.

In certain example embodiments, the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

In certain example embodiments, the amphiphilic cellulose derivative comprises a number-average molecular weight (Mn) of 15,000 g/mol or greater.

In certain example embodiments, the amphiphilic cellulose derivative is soluble in an organic solvent, optionally chosen from an alcohol, tetrahydrofuran, chloroform, acetone, dimethyl sulfoxide, dimethylformamide, dimethylacetamide, and any combination thereof.

In certain example embodiments, the amphiphilic cellulose derivative is water soluble at 50 mg/mL or greater.

In certain example embodiments, the amphiphilic cellulose derivative exhibits a surface tension of 35 to 70 mN/m or 60 mN/m or less, optionally 50 mN/m or less, optionally 40 mN/m or less.

In certain example embodiments, the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative.

In certain example embodiments, the nucleation induction time is increased about 2 to about 45 fold.

In certain example embodiments, the amphiphilic cellulose derivative exhibits a surface tension 50 mN/m or less, preferably 40 mN/m or less, and wherein the nucleation induction time is increased about 2 to 45 fold or 12 to 45 fold.

In certain example embodiments, the nucleation induction time of the supersaturated aqueous solution is greater than 0 minutes but less than 25 minutes or less than 10 minutes.

Described in certain example embodiments herein are amorphous solid dispersions comprising an amphiphilic cellulose derivative of the present disclosure; and one or more agents, optionally wherein one or more of the one or more agents is poorly water soluble.

Described in certain example embodiments are amorphous solid dispersions comprising: a cellulose or a cellulose derivative comprising one or more α,ω-dicarboxylic acid mono-ester (“ω-carboxyalkanoate ester”) substituents; and one or more agents, optionally wherein one or more of the one or more agents is poorly water soluble. In some embodiments, the one or more agents are crystalline organic molecule(s).

In certain example embodiments, the one or more agents are each independently selected from a weakly basic pharmaceutical compound, a neutral pharmaceutical compound, or a weakly acidic pharmaceutical compound.

In certain example embodiments one or more of the one or more agents is a therapeutic agent.

In certain example embodiments, one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof.

In certain example embodiments, the one or more agents are each independently selected from nonsteroidal anti-inflammatory agents (NSAIDs), antifungal agents, chemotherapeutics, or any combination thereof. In certain example embodiments, the one or more agents are each independently selected from Celecoxib, Enzalutamide, Posaconazole, or any combination thereof.

In certain example embodiments, the one or more agents, optionally one or more of the poorly water soluble agents, has increased bioavailability as compared to its free form.

In certain example embodiments, the one or more agents, optionally one or more of the poorly soluble agents, has increased oral bioavailability as compared to its free form.

Described certain example embodiments herein, the amorphous solid dispersion is prepared by a method comprising reacting a cellulose or cellulose derivative and an aliphatic cyclic anhydride, wherein reacting results in the ω-carboxyalkanoate ester substituent of the amphiphilic cellulose derivative.

In certain example embodiments, the aliphatic cyclic anhydride is a succinic anhydride, glutaric anhydride, or a substituted derivative thereof, optionally wherein the derivative thereof comprises an alkanoate substituent, an alkyl substituent, a hydroxyalkyl substituent, or any combination thereof. In certain example embodiments, reacting comprises a ring opening reaction. In certain example embodiments, the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof.

In certain example embodiments, the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents. In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxypropyl cellulose, hydroxyethyl cellulose, or any combination thereof.

In certain example embodiments, the amphiphilic cellulose derivative does not comprise oligomeric anhydrides, poly(anhydrides), side chains thereof, or any combination thereof, optionally crosslinked or capable of forming crosslinks between hydroxyl groups of different molecules of the amphiphilic cellulose derivative; and/or wherein the method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both.

In certain example embodiments, one or more side reactions are eliminated, reduced, minimized, or any combination thereof by optimizing one or more reaction parameters, optionally wherein the one or more reaction parameters is reaction time, reaction temperature, stoichiometry, solvent, product isolation, product isolation, or any combination thereof.

In certain example embodiments, the amorphous solid dispersion is prepared by a method comprising dissolving the one or more agents, optionally one or more poorly water soluble agents, and one or more amphiphilic cellulose derivatives in one or more organic solvent(s), optionally chosen from an alcohol, tetrahydrofuran, chloroform, acetone, dimethyl sulfoxide, dimethylformamide, and dimethylacetamide; and removing the solvent from one or more agents and the one or more amphiphilic cellulose derivatives, thus forming the amorphous solid dispersion.

In certain example embodiments, the amorphous solid dispersion is prepared by thermal extrusion of one or more amphiphilic cellulose derivatives and one or more agents, optionally one or more therapeutic agents, so as to form the amorphous solid dispersion. In certain example embodiments the amorphous solid dispersion is prepared by electrospinning of the one or more amphiphilic cellulose derivatives and one or more agents, optionally one or more therapeutic agents, so as to form the amorphous solid dispersion.

In certain example embodiments, one or more of the one or more amphiphilic cellulose derivatives comprises one or more ω-carboxyalkanoate ester substituents.

In some embodiments, the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents. In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the amphiphilic cellulose derivative comprises acetate substituents, propionate substituents, butyrate substituents, hydroxyalkyl ether substituents, hydroxy functional polyether substituents, or combinations thereof. In certain example embodiments, the amphiphilic cellulose derivative is MCC succinate, CA320S succinate (low DS), CA320S (high DS), CA320S glutarate, CA398 succinate, cellulose acetate butyrate (CAB-553-0.4) succinate, or cellulose acetate propionate (CAP-504-0.2) glutarate.

In certain example embodiments, the amphiphilic cellulose derivative comprises a number-average molecular weight (Mn) of 15,000 g/mol or greater.

In certain example embodiments, the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

In certain example embodiments, the amphiphilic cellulose derivative is water soluble at 50 mg/mL or greater.

In certain example embodiments, the amphiphilic cellulose derivative exhibits a surface tension of 35 to 70 mN/m.

In certain example embodiments, the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold.

In certain example embodiments, the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of the supersaturated aqueous solution is greater than 0 minutes but less than 25 minutes or less than 10 minutes.

In certain example embodiments, the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold.

In certain example embodiments, the one or more amphiphilic cellulose derivatives are prepared by a method comprising mono-esterification of a plurality of diacids or derivatives, with a plurality of hydroxyl groups of a cellulose or a cellulose derivative.

In certain example embodiments, each of the plurality of diacids are independently dicarboxylic acids, optionally α,ω-dicarboxylic acids, or more optionally C₄ or higher α,ω-dicarboxylic acids.

In certain example embodiments, the dicarboxylic acids are each independently selected from succinic acid, glutaric acid any substituted derivative thereof, and any combination thereof, optionally wherein the derivative thereof comprises an alkanoate substituent, an alkyl substituent, a hydroxyalkyl substituent, or any combination thereof.

In certain example embodiments, the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides.

In certain example embodiments, the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

Described in certain example embodiments here are pharmaceutical formulations comprising an amorphous solid dispersion of the present disclosure. In certain example embodiments, the one or more agents, optionally one or more of the poorly soluble agents, has increased oral bioavailability as compared to the free form of the one or more agents. In certain example embodiments, the amorphous solid dispersion is in the form of granulated particles or a tablet.

Described in certain example embodiments herein are kits comprising an amorphous solid dispersion of the present disclosure or a pharmaceutical formulation thereof, optionally a pharmaceutical formulation of the present disclosure.

Described in certain example embodiments herein are methods of delivering one or more agents to a subject in need thereof, the method comprising administering to the subject in need thereof an amorphous solid dispersion of the present disclosure or a pharmaceutical formulation thereof, optionally a pharmaceutical formulation of the present disclosure.

In certain example embodiments, the amorphous solid dispersion or pharmaceutical formulation thereof is effective to treat or prevent a disease, condition, disorder, or a symptom thereof in the subject.

Described in certain example embodiments herein are methods of preparing amphiphilic cellulose derivatives and/or an amorphous solid dispersion thereof comprising one or more α,ω-dicarboxylic acid mono-ester (“ω-carboxyalkanoate ester”) substituents, the method comprising reacting a cellulose or cellulose derivative and an aliphatic cyclic anhydride, wherein reacting results in the ω-carboxyalkanoate ester substituents of the amphiphilic cellulose derivative.

In certain example embodiments, the aliphatic cyclic anhydride is a succinic anhydride, glutaric anhydride, a substituted derivative thereof, or any combination thereof, optionally wherein the derivative thereof comprises an alkanoate substituent, an alkyl substituent, a hydroxyalkyl substituent, or any combination thereof.

In certain example embodiments, reacting comprises a ring opening reaction.

In certain example embodiments, the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof.

In certain example embodiments, the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents. In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxyethyl cellulose, or any combination thereof.

In certain example embodiments, wherein the method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both.

In certain example embodiments, the side reactions are eliminated, reduced, or minimized by optimizing one or more reaction parameters, optionally wherein the one or more reaction parameters is reaction time, reaction temperature, stoichiometry, solvent, product isolation, product isolation, or any combination thereof.

Described in certain example embodiments herein are methods of preparing an amphiphilic cellulose derivative, the method comprising mono-esterification of a plurality of diacids or derivatives with a plurality of hydroxyl groups of a cellulose or a cellulose derivative, optionally wherein the plurality of diacids or derivates thereof comprise anhydrides.

In certain example embodiments, the amphiphilic cellulose derivative is capable of inhibiting crystallization of one or more agents, optionally one or more poorly water soluble agents, from aqueous solution.

In certain example embodiments, the method mono-esterifies an average of at least 50% of the available hydroxyl groups of a cellulose or a cellulose derivative.

In certain example embodiments, each of the plurality of diacids are independently dicarboxylic acids, optionally α,ω-dicarboxylic acids, or more optionally C₄ or higher α,ω-dicarboxylic acids.

In certain example embodiments, the dicarboxylic acids are each independently selected from succinic acid, glutaric acid, a substituted derivative thereof, and any combination thereof, optionally wherein the derivative thereof comprises an alkanoate substituent, an alkyl substituent, a hydroxyalkyl substituent, and any combination thereof.

Described in certain example embodiments herein are methods of preparing an amphiphilic cellulose derivative, the method comprising: mono-esterification of a plurality of diacids or derivatives with a plurality of hydroxyl groups of a cellulose or a cellulose derivative, optionally wherein the plurality of diacids or derivates thereof comprise anhydrides.

In certain example embodiments, the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides.

In certain example embodiments, the diacids are dicarboxylic acids and are each independently chosen from succinic acid and glutaric acid.

In certain example embodiments, the cellulose is microcrystalline cellulose.

In certain example embodiments, the cellulose derivative comprises an average degree of substitution of available hydroxyl groups of 1.8 or greater.

In certain example embodiments, the cellulose derivative comprises ester substituents having the formula —C(═O)R, ether substituents having the formula —OR, or combinations thereof. In certain example embodiments, the cellulose derivative comprises ester substituents having the formula —C(═O)R, ether substituents having the formula —OR, or any combination thereof, optionally wherein R is independently chosen at each occurrence from —OH and an alkyl. In certain example embodiments, R is independently chosen from hydroxyl groups, alkyl ester substituents, and alkoxide ester substituents.

In certain example embodiments, the cellulose derivative comprises one or more acetate substituents, one or more propionate substituents, one or more butyrate substituents, one or more hydroxyalkyl substituents, one or more hydroxy functional polyether substituents, or any combination thereof.

In certain example embodiments, the cellulose derivative is cellulose acetate, cellulose acetate butyrate, cellulose acetate propionate, hydroxypropyl cellulose, or hydroxyethyl cellulose.

In certain example embodiments, the amphiphilic cellulose derivative comprises a number-average molecular weight (Mn) of 15,000 g/mol or greater.

In certain example embodiments, the amphiphilic cellulose derivative is soluble in one or more organic solvents, wherein the one or more organic solvents are optionally chosen from an alcohol, tetrahydrofuran, chloroform, acetone, dimethyl sulfoxide, dimethylformamide, and dimethylacetamide.

In certain example embodiments, the amphiphilic cellulose derivative is water soluble at 50 mg/mL or greater.

In certain example embodiments, the amphiphilic cellulose derivative exhibits a surface tension of 35 to 70 mN/m.

In certain example embodiments, the amphiphilic cellulose derivative is capable of increasing nucleation induction time of a supersaturated aqueous solution comprising the amphiphilic cellulose derivative and one or more agents as compared to a solution without the amphiphilic cellulose derivative.

In certain example embodiments, the nucleation induction time is increased about 2 to about 45 fold.

In certain example embodiments, the amphiphilic cellulose derivative exhibits a surface tension of 50 mN/m or less, optionally less than 40 mN/m, and is capable of increasing nucleation induction time of the supersaturated aqueous solution 2 to 45 fold or 12 to 45 fold.

In certain example embodiments, the amphiphilic cellulose derivative is water soluble.

In certain example embodiments, the amphiphilic cellulose derivative comprises an average of at least 25% carboxylic acid functional substituents based on total constituents.

In certain example embodiments, the amphiphilic cellulose derivative exhibits a surface tension of 60 mN/m or less, preferably less than 50 mN/m.

In certain example embodiments, the supersaturated aqueous solution comprises a loading ratio of the one or more agents, optionally one or more poorly soluble agents, and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of the supersaturated aqueous solution is 25 minutes or less or 10 minutes or less.

Described in certain example embodiments herein are methods of forming an amorphous solid dispersion (ASD) comprising one or more agents, optionally one or more poorly soluble agents, the method comprising the steps of dissolving the one or more agents, optionally one or more poorly soluble agents, and one or more amphiphilic cellulose derivatives in one or more organic solvent(s), optionally chosen from an alcohol, tetrahydrofuran, chloroform, acetone, dimethylsulfoxide, dimethylformamide, and dimethylacetamide; and removing the solvent from one or more agents, optionally one or more poorly soluble agents, and the one or more amphiphilic cellulose derivatives, thus forming the amorphous solid dispersion.

Described in certain example embodiments herein are methods of forming an amorphous solid dispersion (ASD) comprising thermal extrusion of one or more amphiphilic cellulose derivatives and one or more agents, optionally one or more therapeutic agents, so as to form the amorphous solid dispersion. Described in certain example embodiments herein are methods of forming an amorphous solid dispersion (ASD) by electrospinning of the one or more amphiphilic cellulose derivatives and one or more agents, optionally one or more therapeutic agents, so as to form the amorphous solid dispersion.

In certain example embodiments, one or more of the one or more amphiphilic cellulose derivatives comprises one or more ω-carboxyalkanoate ester substituents.

In certain example embodiments, the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents. In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the amphiphilic cellulose derivative comprises acetate substituents, propionate substituents, butyrate substituents, hydroxy functional polyether substituents, or combinations thereof.

In certain example embodiments, the amphiphilic cellulose derivative is MCC succinate, CA320S succinate (low DS), CA320S (high DS), CA320S glutarate, CA398 Succinate, cellulose acetate butyrate (CAB-553-0.4) succinate, or cellulose acetate propionate (CAP-504-0.2) glutarate.

In certain example embodiments, the amphiphilic cellulose derivative comprises a number-average molecular weight (Mn) of 15,000 g/mol or greater.

In certain example embodiments, the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

In certain example embodiments, the amphiphilic cellulose derivative is water soluble at 50 mg/mL or greater.

In certain example embodiments, the amphiphilic cellulose derivative exhibits a surface tension of 35 to 70 mN/m.

In certain example embodiments, the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold.

In certain example embodiments, the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of the supersaturated aqueous solution is greater than 0 minutes but less than 25 minutes or less than 10 minutes.

In certain example embodiments, the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold.

In certain example embodiments, the method further comprises grinding the amorphous solid dispersion to a desired particle size, and optionally compressing the amorphous solid dispersion particles into tablets.

In certain example embodiments, the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the one or more agents is/are crystalline organic molecule(s).

In certain example embodiments, the one or more agents are each independently selected from a weakly basic pharmaceutical compound, a neutral pharmaceutical compound, or a weakly acidic pharmaceutical compound.

In certain example embodiments, one or more of the one or more agents is a therapeutic agent.

In certain example embodiments, one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof.

In certain example embodiments, the one or more agents are each independently selected from nonsteroidal anti-inflammatory agents (NSAIDs), antifungal agents, chemotherapeutics, or any combination thereof.

In certain example embodiments, the one or more agents are each independently selected from Celecoxib, Enzalutamide, Posaconazole, or any combination thereof.

Described in certain example embodiments herein are amphiphilic cellulose derivatives comprising one or more ω-carboxyalkanoate ester substituents, wherein the amphiphilic cellulose derivative is made by a method as described herein.

In certain example embodiments, the amphiphilic cellulose derivative is MCC succinate, CA320S succinate (low DS), CA320S (high DS), CA320S glutarate, CA398 succinate, cellulose acetate butyrate (CAB-553-0.4) succinate, or cellulose acetate propionate (CAP-504-0.2) glutarate.

Described in certain example embodiments herein are amphiphilic cellulose derivatives prepared by the method of described herein. In certain example embodiments, the amphiphilic cellulose derivatives comprise one or more ω-carboxyalkanoate ester substituents.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a strategy for evaluating nucleation time of the cellulose ester derivative for amorphous solid dispersions.

FIG. 2 shows results for nucleation time of the exemplary weakly acidic agent, celecoxib.

FIG. 3 shows results for nucleation time of the exemplary weakly basic agent, posaconazole.

FIG. 4 shows results for nucleation time of the exemplary neutral agent, enzalutamide.

FIG. 5 shows results demonstrating the relationship between surface tension and induction time.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains.

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets (including but not limited to canines, felines, equines, avians, bovines, camelids, ovines, swine, and/or the like). Tissues, cells and their progeny of a biological entity obtained in vivo or cultured in vitro are also encompassed.

As used herein, “administering” refers to the act of delivering a substance to a subject by any suitable methods, techniques, or routes. Administration routes, include, but are not limited to, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease or condition to be treated/prevented, one or more characteristics of the subject, the compositions/substance being delivered, or a combination thereof. One of ordinary skill in the art, unless otherwise noted herein, will appreciate such parameters and be capable of determine a suitable delivery technique and route.

As used herein, “substituted,” refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups can be further substituted. “Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-30 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups can be further substituted.

As used herein, “alkyl,” refers to the radical of saturated aliphatic groups, including straight-chain alkyl, alkenyl, or alkynyl groups, branched-chain alkyl, cycloalkyl (alicyclic), alkyl substituted cycloalkyl groups, and cycloalkyl substituted alkyl. In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. In some embodiments, preferred alkyl groups are lower alkyls. In preferred embodiments, a substituent designated herein as alkyl is a lower alkyl.

As used herein, “Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CON(R)₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, haloalkyl (such as —CF₃, —CH₂—CF₃, —CCl₃); —CN; —NCOCOCH₂CH₂, —NCOCOCHCH; —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

As used herein, “alkenyl” and “alkynyl” refer to unsaturated aliphatic groups analogous in length and possible substitution to the alkyls described above, but that contain at least one double or triple bond, respectively. The term “substituted alkenyl” refers to alkenyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. The term “substituted alkynyl” refers to alkynyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

As used herein, “alkoxyl” or “alkoxy,” “aroxy” or “aryloxy,” generally describe compounds represented by the formula —OR^{v}, wherein R^{v} includes, but is not limited to, substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl. The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. The term alkoxy also includes cycloalkyl, heterocyclyl, cycloalkenyl, heterocycloalkenyl, and arylalkyl having an oxygen radical attached to at least one of the carbon atoms, as valency permits.

As used herein, “amino” and “amine,” are art-recognized and refer to both substituted and unsubstituted amines, e.g., a moiety that can be represented by the general formula:

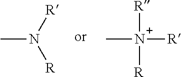

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(CH₂)ₘ—R′″. Thus, the term ‘alkylamine’ as used herein refers to an amine group, as defined above, having a substituted or unsubstituted alkyl attached thereto (i.e., at least one of R, R′, or R″ is an alkyl group).

As used herein, “arylalkyl,” refers to an alkyl group that is substituted with a substituted or unsubstituted aryl or heteroaryl group.

As used herein, “alkylaryl,” as used herein, refers to an aryl group (e.g., an aromatic or hetero aromatic group), substituted with a substituted or unsubstituted alkyl group.

As used interchangeably herein, the terms “amide” or “amido” refer to both “unsubstituted amido” and “substituted amido” and are represented by the general formula:

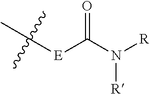

wherein, E is absent, or E is substituted or unsubstituted alkyl, substituted or unsubstituted alkenyl, substituted or unsubstituted alkynyl, substituted or unsubstituted aralkyl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ each independently represent a hydrogen atom, substituted or unsubstituted alkyl, a substituted or unsubstituted alkenyl, or —(CH₂)ₘ—R′″. When E is oxygen, a carbamate is formed. The carbamate cannot be attached to another chemical species, such as to form an oxygen-oxygen bond, or other unstable bonds, as understood by one of ordinary skill in the art.

As used herein, “arylthio” refers to —S-aryl or —S-heteroaryl groups, wherein aryl and heteroaryl are as defined herein. The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein. The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

As used herein, “aryl” refers to C₅-C₂₆-membered aromatic, fused aromatic, fused heterocyclic, or biaromatic ring systems. Broadly defined, “aryl,” as used herein, includes 5-, 6-, 7-, 8-, 9-, 10-, 14-, 18-, and 24-membered single-ring aromatic groups, for example, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

As used herein, “carbonyl,” is art-recognized and includes such moieties as can be represented by the general formula:

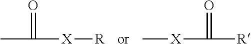

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defined as above, the moiety is also referred to as a carboxyl group. When X is oxygen and R is hydrogen, the formula represents a ‘carboxylic acid’. Where X is oxygen and R′ is hydrogen, the formula represents a ‘formate’. Where X is oxygen and R or R′ is not hydrogen, the formula represents an “ester”. In general, where the oxygen atom of the above formula is replaced by a sulfur atom, the formula represents a ‘thiocarbonyl’ group. Where X is sulfur and R or R′ is not hydrogen, the formula represents a ‘thioester.’ Where X is sulfur and R is hydrogen, the formula represents a ‘thiocarboxylic acid.’ Where X is sulfur and R′ is hydrogen, the formula represents a ‘thioformate.’ Where X is a bond and R is not hydrogen, the above formula represents a ‘ketone.’ Where X is a bond and R is hydrogen, the above formula represents an ‘aldehyde.’

The term “substituted carbonyl” refers to a carbonyl, as defined above, wherein one or more hydrogen atoms in R, R′ or a group to which the moiety

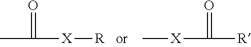

is attached, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “carboxyl” is as defined above for the formula

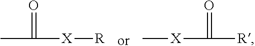

and is defined more specifically by the formula —R^{iv}COOH, wherein R^{iv} is an alkyl, alkenyl, alkynyl, cycloalkyl, heterocyclyl, alkylaryl, arylalkyl, aryl, or heteroaryl. In preferred embodiments, a straight chain or branched chain alkyl, alkenyl, and alkynyl have 30 or fewer carbon atoms in its backbone (e.g., C₁-C₃₀ for straight chain alkyl, C₃-C₃₀ for branched chain alkyl, C₂-C₃₀ for straight chain alkenyl and alkynyl, C₃-C₃₀ for branched chain alkenyl and alkynyl), preferably 20 or fewer, more preferably 15 or fewer, most preferably 10 or fewer. Likewise, preferred cycloalkyls, heterocyclyls, aryls and heteroaryls have from 3-10 carbon atoms in their ring structure, and more preferably have 5, 6 or 7 carbons in the ring structure. The term “substituted carboxyl” refers to a carboxyl, as defined above, wherein one or more hydrogen atoms in R are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

As used herein, the terms “weight percent,” “wt %,” and “wt. %,” which are used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “derivative” refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” includes prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

As used interchangeably herein, “heterocycle,” “heterocyclic” and “heterocyclyl” refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

As used herein, “heteroaryl” refers to C₅-C₂₆-membered aromatic, fused aromatic, biaromatic ring systems, or combinations thereof, in which one or more carbon atoms on one or more aromatic ring structures have been substituted with an heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined for “substituted heteroaryl”. The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

As used herein, “heteroalkyl,” refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

As used herein, “anti-infective” refers to compounds or molecules that can either kill an infectious agent or inhibit it from spreading. Anti-infectives include, but are not limited to, antibiotics, antibacterials, antifungals, antivirals, and antiprotozoals.

As used herein, “chemotherapeutic agent” or “chemotherapeutic” refers to a therapeutic agent utilized to prevent or treat cancer.

As used herein, “immunomodulator,” refers to an agent, such as a therapeutic agent, which is capable of modulating or regulating one or more immune function or response.

As used herein, “control” can refer to an alternative subject or sample used in an experiment for comparison purpose and included to minimize or distinguish the effect of variables other than an independent variable.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “therapeutic” refers to preventing, treating, healing, and/or ameliorating a disease, disorder, condition, or side effect, or to decreasing in the rate of advancement of a disease, disorder, condition, or side effect. A “therapeutically effective amount” therefore refers to an amount of a compound that can yield a therapeutic effect.

As used herein, the terms “treating” and “treatment” refers generally to obtaining a desired pharmacological and/or physiological effect. The effect can be, but does not necessarily have to be, prophylactic in terms of preventing or partially preventing a disease, symptom or condition thereof. effect can be therapeutic in terms of a partial or complete cure of a disease, condition, symptom or adverse effect attributed to the disease, disorder, or condition. The term “treatment” as used herein covers any treatment of a disease in a subject, particularly a human, and includes any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, includes inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

As used herein, “agent” refers to any substance, compound, molecule, and the like, which can be biologically active or otherwise can induce a biological and/or physiological effect on a subject to which it is administered to.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “substantial” and “substantially,” specify an amount of between 95% and 100%, inclusive, between 96% and 100%, inclusive, between 97% and 100%, inclusive, between 98% 100%, inclusive, or between 99% 100%, inclusive.

As used herein, “solubility” refers to the phenomenon of dissolution of a solute in a solvent to yield a homogenous or substantially homogenous system, and more particularly, the property of a solid, liquid, or gaseous chemical substance called solute to dissolve in a solid, liquid, or gaseous solvent to form a homogeneous solution of the solute in the solvent. The extent of solubility of a substance in a specific solvent is measured as the saturation concentration where adding more solute does not increase its concentration in the solution (Lachman L, Lieberman H, Kanig J L. The Theory And Practise of Industrial Pharmacy. 3rd edition. Lea & Febiger; 1986). Solubility occurs under dynamic equilibrium, which means that solubility results from the simultaneous and opposing processes of dissolution and phase joining (e.g., precipitation of solids). Solubility equilibrium occurs when the two processes proceed at a constant rate. Under certain conditions equilibrium solubility may be exceeded to give a so-called supersaturated solution, which is metastable (Myrdal P B, Yalkowsky S H. Solubilization of drugs in aqueous media. In: Swarbrick J, editor. Encyclopedia of Pharmaceutical Technology. 3rd edition. New York, NY, USA: Informa Health Care; 2007. p. p. 3311). IUPAC defines solubility as the analytical composition of a saturated solution expressed as a proportion of a designated solute in a designated solvent, which is encompassed by the definition herein. Solubility can be stated in units of concentration, molality, mole fraction, mole ratio, and other units (IUPAC gold book. http://goldbook.iupac.org/S05740.html). In the context of agents or compounds for pharmaceutical use, the solvent for determining the solubility of a compound or agents is water. The solubility of any particular compound or agent can be classified according to the USP and BP solubility criteria regardless of solvent, just only in terms of quantification (The United States Pharmacopeia, USP 30-NF 25, 2007 and British Pharmacopoeia, 2009) as shown in the Table below.

TABLE 1
USP and BP solubility criteria

| Descriptive Term | Part of solvent required per part of solute |
|---|---|
| Very soluble | Less than 1 |
| Freely soluble | From 1 to 10 |
| Soluble | From over 10 to 30 |
| Sparingly soluble | From over 30 to 100 |
| Slightly soluble | From over 100 to 1000 |
| Very slightly soluble | From over 1000 to 10,000 |
| Practically soluble | Over 10,000 |

In the context of pharmaceutical compounds or agents, solubility can be classified or predicted based on the Biopharmaceutics Classification System (BCS) provided by the U.S. Food and Drug Administration. Solubility in this context is based on the highest-dose strength of an immediate release product. drug is considered highly soluble when the highest dose strength is soluble in 250 mL or less of aqueous media over the pH range of 1 to 7.5. The volume estimate of 250 mL is derived from typical bioequivalence study protocols that prescribe administration of a drug product to fasting human volunteers with a glass of water (Amidon G L, Lennernfs H, Shah V P, Crison J R. A theoretical basis for a biopharmaceutic drug classification: the correlation of in vitro drug product dissolution and in vivo bioavailability. Pharmaceutical Research. 1995; 12(3):413-420). All drug compounds can be classified in to four classes under this system: class I—high soluble and high permeable, class II—low soluble and high permeable, class III—high soluble and low permeable and class IV—low soluble and low permeable. Poorly soluble compounds or agents herein can fall into either Class II or IV. The poorly soluble compounds or agents can be poorly water soluble.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Drug solubilization is an essential step for orally administered medications to be absorbed systemically. However, an estimated forty percent of approved therapeutics and an estimated ninety percent of those in the research and development pipeline are poorly water-soluble. See e.g., Babu and Nangia. Cryst Growth Des, 11 (2011), 2662-2679; Benet et al., AAPS J. 13(2011) 519-547; and Jatwani et al., Int J Pharma Sci Res 3(2012) 942. Various strategies have been employed to overcome issues of solubility and/or dissolution rate associated with poorly water-soluble drugs, including amorphous solid dispersion (ASD).

ASD is a solid dispersion in which the active ingredient is dispersed within an excipient matrix in a substantially amorphous form (Chiou and Riegelman. J Pharm Sci. 1970. 1281-1302). The amorphous state of the agent is important for increasing its solubility. When an agent is in an amorphous form, no energy is needed to break the agent crystal lattice. As such, relative to the crystalline form of the agent, the amorphous form of many poorly water soluble agents can achieve substantially greater apparent solubility and markedly faster dissolution. ASDs can also result in greater membrane flux due to a higher supersaturation, and thus also have improved bioavailability.

Surface tension is a property that quantifies the force per unit length acting at the surface of a liquid and is typically measured in units of force per unit length (e.g., N/m or dyn/cm). The surface tension of a polymer can impact various aspects of ASD formulation and performance. For example, a hydrophilic or amphiphilic polymer with lower surface tension can enhance the wetting and spreading of the dispersion on solid drug particles, facilitating dissolution and drug release. Lower surface tension can also contribute to improved dispersibility and uniform coating of the amorphous solid dispersion during processing. Wettability of ASDs can also be greater when in the presence of hydrophilic or amphiphilic polymers. As used herein, unless otherwise indicated, the term “induction time” refers to the period during which the amorphous drug remains stable and does not undergo recrystallization or transformation back into its crystalline form. It is a critical parameter in the formulation of amorphous solid dispersions as it determines the stability and performance of the formulation. When a drug is converted from its crystalline form to an amorphous form, it gains higher energy and a less thermodynamically stable state. As a result, there is a natural tendency for the drug molecules to reorganize and recrystallize, returning to their more stable crystalline state. The induction time represents the duration during which the amorphous drug remains in a metastable state before recrystallization occurs. The length of the induction time depends on various factors, including the drug and polymer properties, the formulation composition, and storage conditions. Factors that can influence the induction time include molecular mobility, glass transition temperature (Tg), drug-polymer interactions, drug concentration, and the presence of stabilizers.

Several techniques can be employed to extend the induction time and improve the stability of amorphous solid dispersions. These include the use of appropriate polymers that can inhibit drug recrystallization, the addition of stabilizers or anti-crystallization agents, and controlling the formulation composition and processing conditions. It is important to understand and evaluate the induction time of amorphous solid dispersions during formulation development and stability testing. Techniques such as differential scanning calorimetry (DSC), X-ray diffraction (XRD), and dissolution testing can be employed to assess the induction time and monitor any recrystallization or phase transformation processes that may occur over time. By optimizing the formulation and understanding the factors influencing the induction time, it is possible to design stable amorphous solid dispersions with prolonged shelf life and improved bioavailability, ensuring the therapeutic efficacy of poorly soluble drugs.

With that said, embodiments disclosed herein can provide amphiphilic cellulose derivatives and ASD preparations with amphiphilic cellulose derivatives. Without being bound by theory the synthetic methods described herein can overcome significant synthetic barriers with ASD preparations and provide amphiphilic cellulose derivates that are superior in generating and maintaining supersaturated solutions of various therapeutically relevant compounds. Also described in exemplary embodiments herein are methods of making and using the ASD formulations, particularly for improving the solubility and dissolution of therapeutically relevant, yet poorly water soluble, compounds. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Preparing Cellulose Derivatives

Described in embodiments herein are methods of preparing cellulose derivatives, particularly amphiphilic cellulose derivatives, suitable for preparing amorphous solid dispersions (ASDs). Without being bound by theory, such preparations can be advantageous for preparing amorphous solid dispersions of agents that are poorly water soluble and increase bioavailability of such agents. In some embodiments, the methods allow avoidance of certain side reactions, such as homopolymerization of the cyclic anhydrides, as well as crosslinking of the products due to growth of oligo(anhydride) side chains from cellulose, followed by reaction of these oligo(anhydrides) with residual cellulose hydroxyls on another polymer chain. Further, certain embodiments of the methods herein allow for simple, one-step synthesis of cellulose ω-carboxyalkanoates that also contain alkanoate, alkyl, or hydroxyalkyl substituents, from inexpensive cellulose derivatives.

In certain embodiments, the methods produce cellulose derivatives that contain ω-carboxyalkanoate ester substituents where the ω-carboxyalkanoate substituent arises by ring opening of succinic anhydride, glutaric anhydride, or substituted derivatives thereof. These derivatives may also contain alkanoate, alkyl, or hydroxyalkyl substituents.

In certain embodiments, described herein are processes for manufacturing cellulose derivatives that contain ω-carboxyalkanoate ester substituents that involves reacting cellulose, cellulose alkanoates, alkyl cellulose ethers, hydroxyalkyl cellulose ethers with cyclic anhydrides including succinic anhydride, glutaric anhydride, or combinations thereof, or derivatives of succinic or glutaric anhydrides. In some embodiments, conditions (including reaction time, temperature, stoichiometry, solvent, and product isolation) are carefully controlled and/or optimized and reduce and/or eliminate side reactions including homopolymerization of the cyclic anhydride, and chain extension of the originally formed ω-carboxyalkanoate to an oligomeric poly(anhydride) side chain of cellulose, that can form crosslinks via reaction with hydroxyl groups on other cellulose derivative molecules.

These and other exemplary embodiments are further described below.

Described in certain example embodiments herein are methods of preparing an amphiphilic cellulose derivative, the method comprising mono-esterification of a plurality of diacids or derivatives with a plurality of hydroxyl groups of a cellulose or a cellulose derivative, optionally wherein the plurality of diacids or derivates thereof comprise anhydrides.

In certain example embodiments, the amphiphilic cellulose derivative is capable of inhibiting crystallization of one or more agents, optionally one or more poorly water soluble agents, from aqueous solution.

In certain example embodiments, the method mono-esterifies an average of at least 50% of the available hydroxyl groups of a cellulose or a cellulose derivative.

In certain example embodiments, each of the plurality of diacids are independently dicarboxylic acids, optionally α,ω-dicarboxylic acids, or more optionally C₄ or higher α,ω-dicarboxylic acids.

In certain example embodiments, the dicarboxylic acids are each independently selected from succinic acid or glutaric acid.

In certain example embodiments, the plurality of diacid derivatives are anhydrides, optionally wherein the anhydrides are independently carboxylic anhydrides, optionally linear or cyclic anhydrides, optionally C₄-C₈ or higher linear or cyclic anhydrides. In some embodiments, the anhydrides are C₄ or C₅ anhydrides. In some embodiments, the anhydrides are C₄ or C₅ linear or cyclic anhydrides. In some embodiments, the carboxylic anhydrides are C₄ or C₅ carboxylic anhydrides. In some embodiments, the carboxylic anhydrides are C₄ or C₅ linear or cyclic carboxylic anhydrides.

In certain example embodiments, the dicarboxylic acids are each independently chosen from succinic acid or glutaric acid.

In certain example embodiments, the dicarboxylic acids are each independently chosen from succinic acid, glutaric acid.

In certain example embodiments, the cellulose is microcrystalline cellulose.

In certain example embodiments, the cellulose derivative comprises an average degree of substitution of available hydroxyl groups of 1.8 or greater. In some embodiments, the average degree of substitution of available hydroxyl groups is 1.8, 1.9, 2.0, 2.1, 2.2, 2.3, 2.4, 2.5, or greater.

In certain example embodiments, the cellulose derivative comprises ester substituents having the formula —C(═O)R, ether substituents having the formula —OR, or combinations thereof. In certain example embodiments, R is independently chosen from hydroxyl groups, alkyl ester substituents, and alkoxide ester substituents. In certain example embodiments, wherein R is independently chosen at each occurrence from —OH and an alkyl.

In certain example embodiments, the cellulose derivative comprises one or more acetate substituents, one or more propionate substituents, one or more butyrate substituents, one or more hydroxy functional polyether substituents, or any combination thereof.

In certain example embodiments, the cellulose derivative is cellulose acetate, cellulose acetate butyrate, cellulose acetate propionate, or hydroxyethyl cellulose.

In certain example embodiments, the amphiphilic cellulose derivative comprises a number-average molecular weight (Mₙ) of 15,000 g/mol or greater. In certain example embodiments, the amphiphilic cellulose derivative comprises a number-average molecular weight (Mn) of 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol or greater.

In certain example embodiments, the amphiphilic cellulose derivative is soluble in one or more organic solvents. In some embodiments, the one or more organic solvents are chosen from an alcohol, tetrahydrofuran, chloroform, acetone, dimethyl sulfoxide, dimethylformamide, dimethylacetamide, and any combination thereof.

In certain example embodiments, the amphiphilic cellulose derivative is water soluble at 50 mg/mL or greater. In some embodiments, the amphiphilic cellulose derivative is water soluble at 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, to/or 100 mg/mL.

In certain example embodiments, the amphiphilic cellulose derivative exhibits a surface tension of 35 to 70 mN/m. In certain example embodiments, the amphiphilic cellulose derivative exhibits a surface tension of 60 mN/m or less. In certain example embodiments, the amphiphilic cellulose derivative exhibits a surface tension of 50 mN/m or less. In certain example embodiments, the amphiphilic cellulose derivative exhibits a surface tension of 40 mN/m or less. In certain example embodiments, the amphiphilic cellulose derivative exhibits a surface tension of 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m, 61 mN/m, 62 mN/m, 63 mN/m, 64 mN/m, 65 mN/m, 66 mN/m, 67 mN/m, 68 mN/m, 69 mN/m, to/or 70 mN/m. In certain example embodiments, the amphiphilic cellulose derivative exhibits a surface tension of greater than 0 to 60 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m. In certain example embodiments, the amphiphilic cellulose derivative exhibits a surface tension of greater than 0 to 50 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m. In certain example embodiments, the amphiphilic cellulose derivative exhibits a surface tension of greater than 0 to 40 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m.

In certain example embodiments, the amphiphilic cellulose derivative is capable of increasing nucleation induction time of a supersaturated aqueous solution comprising the amphiphilic cellulose derivative and one or more agents as compared to a solution without the amphiphilic cellulose derivative.

In certain example embodiments, the nucleation induction time is increased about 2 to about 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the amphiphilic cellulose derivative exhibits a surface tension of 50 mN/m or less, optionally less than 40 mN/m, and is capable of increasing nucleation induction time of the supersaturated aqueous solution 2 to 45 fold or 12 to 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold. In certain example embodiments, the nucleation induction time is increased about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the amphiphilic cellulose derivative is water soluble.

In certain example embodiments, the amphiphilic cellulose derivative comprises an average of at least 25% carboxylic acid functional substituents based on total substituents. In some embodiments, the amphiphilic cellulose derivative comprises an average of 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to/or 100% of carboxylic acid functional substituents based on total substituents.

In certain example embodiments, the supersaturated aqueous solution of the one or more agents, optionally one or more poorly soluble agents, and a carboxylic acid functional cellulose derivative comprises loading ratio of 1:99 wt./wt. ratio or greater (e.g., a 1:99 to 50:50 wt./wt. ratio, including all integer ratios therebetween, a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of a supersaturated aqueous solution of the one or more agents, is 25 minutes or less or 10 minutes or less. In some embodiments, the nucleation induction time of the supersaturated aqueous solution is greater than 0 minutes to/or 1, 2, 3, 4, 5, 6, 7, 8, 9, 10, 11, 12, 13, 14, 15, 16, 17, 18, 19, 20, 21, 22, 23, 24 up to 25 minutes. In some embodiments, the nucleation induction time of the supersaturated aqueous solution is greater than 0 minutes to/or 1, 2, 3, 4, 5, 6, 7, 8, 9, up to 10 minutes.

### Methods of Preparing Amorphous Solid Dispersions (ASDS)

Described in embodiments herein are methods of preparing amorphous solid dispersions (ASDs). In an aspect, cellulose derivatives of the present disclosure, e.g., amphiphilic cellulose derivatives, and/or methods of preparing said cellulose derivatives of the present disclosure are suitable for preparing the ASDs. Without being bound by theory, such amphiphilic cellulose derivatives are suitable for preparing amorphous solid dispersions of agents that are poorly water soluble and increase bioavailability of such agents. In some embodiments, the methods allow avoidance of certain side reactions, such as homopolymerization of the cyclic anhydrides, as well as crosslinking of the products due to growth of oligo(anhydride) side chains from cellulose, followed by reaction of these oligo(anhydrides) with residual cellulose hydroxyls on another polymer chain. Further, certain embodiments of the methods herein allow for simple, one-step synthesis of cellulose ω-carboxyalkanoates that also contain alkanoate, alkyl, or hydroxyalkyl substituents, from inexpensive cellulose derivatives.

The methods described herein can be used to form ASDs, which can contain the cellulose derivatives, such as ω-carboxyalkanoate derivatives with, e.g., poorly soluble drugs and/or other active species. Without being bound by theory, such ASDs can provide unexpectedly long duration of supersaturation of those drugs in aqueous solution, due to the unexpectedly excellent ability of these cellulose derivatives, e.g., ω-carboxyalkanoate derivatives, to stabilize these supersaturated solutions against drug crystallization, thereby strongly enhancing bioavailability in the case of orally administered drugs.

These and other exemplary embodiments are further described below.

### Amorphous Solid Dispersion Processes

The process of making amorphous solid dispersions involves the conversion of a drug, optionally a poorly soluble drug, from its crystalline state into an amorphous form and its subsequent dispersion within a polymeric matrix. This combination of drug and polymer improves the dissolution rate and solubility of the drug, leading to enhanced drug absorption and therapeutic efficacy. The first step in the process is the selection of an appropriate polymer that is compatible with the drug and can form a stable dispersion. Polymers commonly used for this purpose include hydroxypropyl methylcellulose (HPMC), polyvinylpyrrolidone (PVP), polyethylene glycol (PEG), and the like. The selected polymer is usually in a solid form, such as a powder or granules.

The drug and polymer are then mixed together using various techniques to obtain a homogeneous mixture. One commonly employed mixing method is the solvent evaporation method. In this approach, the drug and polymer are dissolved in a common solvent, such as ethanol, methanol, or the like, to form a solution. The solvent is then evaporated under controlled conditions, such as reduced pressure or elevated temperature, resulting in the formation of an amorphous solid dispersion. There are several solvent evaporation methods commonly used for the preparation of ASDs including solvent casting, spray drying, rotary evaporation, solvent spinning, and the like.

In solvent casting, the drug and polymer are dissolved in a suitable solvent, such as ethanol or methanol, to form a homogeneous solution. The solution is then cast onto a flat surface, such as a glass plate, and the solvent is allowed to evaporate slowly at ambient temperature or under controlled conditions like reduced pressure or elevated temperature. The resulting film or sheet is then crushed or ground into a powder, which can be further processed into the desired dosage form.

Spray drying involves the atomization of a solution or suspension of the drug and polymer into fine droplets, which are rapidly dried using hot air or an inert gas stream. The solvent evaporates during the drying process, resulting in the formation of solid particles of the amorphous dispersion. The particles can be collected and further processed, such as by milling or blending, to obtain a suitable formulation.

Rotary evaporation utilizes a rotary evaporator, which is a device that applies vacuum and heat to evaporate solvents from a solution. In this method, the drug and polymer are dissolved in a volatile solvent, and the solution is placed in a round-bottomed flask attached to the rotary evaporator. The flask is rotated under vacuum, and heat is applied to facilitate the evaporation of the solvent. The remaining solid residue in the flask is the amorphous solid dispersion.

Solvent spinning involves dissolving both the drug and the polymer carrier in a common solvent to form a homogeneous solution. This solution is then subjected to spinning using a spinneret or similar equipment. As the solvent is removed during spinning, the drug and polymer precipitate together, forming an amorphous solid dispersion. The rapid evaporation of the solvent can lead to the generation of a highly supersaturated state, favoring the formation of an amorphous structure. The resulting solid dispersion can have improved drug dissolution and bioavailability compared to the crystalline drug form. Solvent spinning can be advantageous as it allows for precise control over the drug-polymer ratios, which can influence the properties of the amorphous solid dispersion. However, it is important to consider the compatibility of the drug, polymer, and solvent to ensure the stability and effectiveness of the resulting formulation.

Another commonly employed mixing method is the melting method, where the drug and polymer are melted together at an elevated temperature to form a molten mixture. The molten mixture is then rapidly cooled to solidify and form an amorphous solid dispersion. This technique is particularly suitable for drugs and polymers with similar melting points. There are several melting methods commonly used for the preparation of ASDs including hot melt extrusion, spinning and the like.

In hot melt extrusion (HME), the drug and polymer are melted together at an elevated temperature, typically using a twin-screw extruder. The molten mixture is then forced through a die, where it solidifies rapidly to form an amorphous solid dispersion. HME is advantageous as it avoids the use of solvents and offers continuous processing capabilities.

Electrospinning can also be employed in the formation of ASDs. Electrospinning is a technique used to produce nanofibers from a polymer solution or melt. It involves applying an electric field to a polymer solution or melt and extruding it through a fine needle or spinneret. The electric field induces a charge on the polymer solution, leading to the formation of a charged jet. As the solvent evaporates during the flight path, solid polymer nanofibers are deposited onto a collector. Electrospun nanofibers have a high surface area-to-volume ratio, interconnected porous structure, and tunable fiber diameter. Electrospinning can be used as a technique to fabricate nanofibers of polymer blends or composite systems, including amorphous solid dispersions. By electrospinning a polymer solution containing both the drug and a polymer carrier, it is possible to obtain nanofibers with a high drug loading and an amorphous state. The process of electrospinning provides unique advantages for the preparation of amorphous solid dispersions, such as high surface area, control over fiber diameter and morphology, and the possibility of incorporating other functional components into the nanofibers. The combination of electrospinning and amorphous solid dispersion offers several benefits in drug delivery applications. The electrospun nanofibers can provide sustained release of the drug due to their high surface area and porous structure. The amorphous solid dispersion within the nanofibers enhances the drug's solubility and dissolution rate, leading to improved drug absorption and therapeutic efficacy. Moreover, the electrospun nanofibers can provide controlled drug release, protection of the drug from degradation, and targeted delivery to specific sites.

Once the amorphous solid dispersion is formed, it undergoes further processing steps to obtain the desired dosage form, such as tablets or capsules. These steps may include granulation, milling, and blending with other excipients to improve flow properties and compressibility. Some of the most common forms of ASDs include: powder, films, granules, tablets, capsules, nanoparticles and the like.

Amorphous solid dispersions can be prepared as powders, which are obtained by methods such as solvent evaporation, spray drying, or milling. These powders can be further processed into tablets, capsules, or incorporated into other dosage forms.

Amorphous solid dispersions can be cast into thin films by solvent casting. The drug-polymer solution is spread onto a flat surface and allowed to dry, resulting in the formation of a thin film. These films can be cut into desired shapes or crushed into powder form for further processing.

Amorphous solid dispersions can be prepared as granules, which are obtained through processes such as wet granulation or melt granulation. In wet granulation, the drug and polymer are dissolved in a solvent, and the solution is sprayed onto a bed of inert particles, followed by drying to form granules. Melt granulation involves melting the drug and polymer mixture, and then solidifying and granulating the melt.

Amorphous solid dispersions can be compressed into tablet form. The ASD powder is blended with excipients such as binders, diluents, and disintegrants, and then compressed into tablets using suitable tablet presses. Tablets offer convenience in dosing and are a common oral dosage form.

Amorphous solid dispersions can be formulated into nanoparticles using techniques like nanoprecipitation, emulsion solvent evaporation, or supercritical fluid techniques. These nanoparticles provide a large surface area for dissolution and can be used for various administration routes, including oral, topical, and parenteral.

In certain cases, the ASDs of the present disclosure can be formed directly in a desired dosage form during the manufacturing process. For example, in the case of hot melt or thermal extrusion, the drug (or active ingredient) and polymer are melted and extruded together to form ASDs which are subsequently shaped into tablets or other forms while in the molten state. In some embodiments, the ASD can be prepared by thermal extrusion of the cellulose ester and active ingredient(s). In certain example embodiments, the amorphous solid dispersion is prepared by electrospinning of the one or more amphiphilic cellulose derivatives and one or more agents, optionally one or more therapeutic agents, so as to form the amorphous solid dispersion. Exemplary Methods of Forming Amorphous Solid Dispersions

Described in certain example embodiments herein are methods of forming an amorphous solid dispersion comprising one or more agents, optionally one or more poorly soluble agents, the method comprising the steps of dissolving the one or more agents, optionally one or more poorly soluble agents, and one or more amphiphilic cellulose derivatives in one or more organic solvent(s), optionally chosen from an alcohol, tetrahydrofuran, chloroform, acetone, dimethyl sulfoxide, dimethylformamide, and dimethylacetamide; and removing the solvent from one or more agents, optionally one or more poorly soluble agents, and the one or more amphiphilic cellulose derivatives, optionally spray drying, thus forming the amorphous solid dispersion.

In certain example embodiments, one or more of the one or more amphiphilic cellulose derivatives comprises one or more ω-carboxyalkanoate ester substituents.

In certain example embodiments, the amphiphilic cellulose derivative comprises ester substituents having the formula —C(═O)R, ether substituents having the formula —OR, or combinations thereof. In some embodiments, R is independently chosen from hydroxyl groups, alkyl ester substituents, and alkoxide ester substituents. In certain example embodiments, R is independently chosen at each occurrence from —OH and an alkyl.

In certain example embodiments, the amphiphilic cellulose derivative comprises acetate substituents, propionate substituents, butyrate substituents, hydroxy functional polyether substituents, or combinations thereof.

In certain example embodiments, the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

In certain example embodiments, the amphiphilic cellulose derivative comprises a number-average molecular weight (Mₙ) of 15,000 g/mol or greater. In certain example embodiments, the amphiphilic cellulose derivative comprises a number-average molecular weight (Mn) of 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol or greater.

In certain example embodiments, the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

In certain example embodiments, the amphiphilic cellulose derivative is water soluble at 50 mg/mL or greater. In certain example embodiments, the amphiphilic cellulose derivative is water soluble at 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL.

In certain example embodiments, the amphiphilic cellulose derivative exhibits a surface tension of 35 to 70 mN/m. In certain example embodiments, the amphiphilic cellulose derivative exhibits a surface tension of 60 mN/m or less. In certain example embodiments, the amphiphilic cellulose derivative exhibits a surface tension of 50 mN/m or less. In certain example embodiments, the amphiphilic cellulose derivative exhibits a surface tension of 40 mN/m or less. In certain example embodiments, the amphiphilic cellulose derivative exhibits a surface tension of 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m, 61 mN/m, 62 mN/m, 63 mN/m, 64 mN/m, 65 mN/m, 66 mN/m, 67 mN/m, 68 mN/m, 69 mN/m, to/or 70 mN/m. In certain example embodiments, the amphiphilic cellulose derivative exhibits a surface tension of greater than 0 to 60 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m. In certain example embodiments, the amphiphilic cellulose derivative exhibits a surface tension of greater than 0 to 50 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m. In certain example embodiments, the amphiphilic cellulose derivative exhibits a surface tension of greater than 0 to 40 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m.

In certain example embodiments, the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the supersaturated aqueous solution of the one or more agents and a carboxylic acid functional cellulose derivative comprises a loading ratio of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of a supersaturated aqueous solution of the one or more agents is greater than 0 minutes but less than 25 minutes or less than 10 minutes. In some embodiments, the nucleation induction time of the supersaturated aqueous solution is greater than 0 minutes to/or 1, 2, 3, 4, 5, 6, 7, 8, 9, 10, 11, 12, 13, 14, 15, 16, 17, 18, 19, 20, 21, 22, 23, 24 up to 25 minutes. In some embodiments, the nucleation induction time of the supersaturated aqueous solution is greater than 0 minutes to/or 1, 2, 3, 4, 5, 6, 7, 8, 9, up to 10 minutes.

In certain example embodiments, the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold. In certain example embodiments, the nucleation induction time is increased about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the method further comprises grinding the amorphous solid dispersion to a desired particle size, and optionally compressing the amorphous solid dispersion particles into tablets.

In certain example embodiments, the one or more agents and the one or more amphiphilic cellulose derivatives are loaded in a 1:99 wt./wt. ratio or greater. In certain example embodiments, the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the one or more agents is/are crystalline organic molecule(s).

In certain example embodiments, the one or more agents are each independently selected from a weakly basic pharmaceutical compound, a neutral pharmaceutical compound, or a weakly acidic pharmaceutical compound.

In certain example embodiments, one or more of the one or more agents is a therapeutic agent.

In certain example embodiments, one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof, or any combination thereof.

In certain example embodiments, the one or more agents are each independently selected from nonsteroidal anti-inflammatory agents (NSAIDs), antifungal agents, chemotherapeutics, or any combination thereof. In certain example embodiments, the one or more agents are each independently selected from Celecoxib, Enzalutamide, Posaconazole, or any combination thereof.

Other exemplary agents that can be included in the ASD preparations provided herein, include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers). In some embodiments, the immunomodulator is an immunosuppressant.

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), temgicoluril, fabomotizole, selank, bromantane, emoxypine, azapirones, barbiturates, hydroxyzine, pregabalin, isovaleric acid, and beta blockers.

Suitable antipsychotics include, but are not limited to, benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatory agents (e.g. ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives (including, but not limited to, anti-bacterials, anti-virals, and anti-fungals) include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/mipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemum, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevacuzimab and thalidomide), NBTXR3, Nimoral, trans sodium crocetinate, NVX-108, and combinations thereof. See also e.g., Kvols, L. K., J Nucl Med 2005; 46:187S-190S.

Described in certain example embodiments herein are amphiphilic cellulose derivatives comprising one or more ω-carboxyalkanoate ester substituents, wherein the amphiphilic cellulose derivative is made by a suitable method a as described elsewhere herein.

In certain example embodiments, the amphiphilic cellulose derivative is MCC succinate, CA320S succinate (low DS), CA320S (high DS), CA320S glutarate, CA398 succinate, cellulose acetate butyrate (CAB-553-0.4) succinate, or cellulose Acetate propionate (CAP-504-0.2) Glutarate. In certain example embodiments, the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

Described in certain example embodiments herein are cellulose esters prepared by a suitable method of any one of the preceding paragraphs or as described elsewhere herein. In certain example embodiments, the cellulose ester comprises one or more ω-carboxyalkanoate ester substituents.

### Amphiphilic Cellulose Derivatives

Described in several exemplary embodiments herein are amphiphilic cellulose derivatives.

Described in certain example embodiments herein are amphiphilic cellulose derivatives comprising a cellulose or a cellulose derivative, wherein one or more hydroxyl groups of the cellulose or the cellulose derivative are replaced by α,ω-dicarboxylic acid mono-ester (“ω-carboxyalkanoate ester”) substituents, where the cellulose or the cellulose derivative is not crosslinked. In some embodiments, the ω-carboxyalkanoate ester is a C₄, C₅, or higher ω-carboxyalkanoate ester substituents, any substituted derivatives thereof. Crosslinking of amphiphilic cellulose derivatives is an issue with current known amphiphilic cellulose derivatives as current methods used to make these compositions result in such crosslinking. Crosslinking between and within amphiphilic cellulose derivatives can impair the functionality of such compounds. Without being bound by theory, the methods of preparing the amphiphilic cellulose derivatives described herein produce am amphiphilic cellulose derivative that is not crosslinked or contains minimal crosslinking so as not to impact the functionality of the amphiphilic cellulose derivatives to form an ASD. Thus, the amphiphilic cellulose derivates of the present disclosure contain a minimal amount of or no crosslinking.

In certain example embodiments, the amphiphilic cellulose derivative does not comprise oligomeric anhydrides, poly(anhydrides), side chains thereof, or any combination thereof, optionally crosslinked or capable of forming crosslinks between hydroxyl groups different molecules of the amphiphilic cellulose derivative.

In certain example embodiments, the ω-carboxyalkanoate ester substituents comprise a terminal carboxylic acid group or a monovalent salt thereof, optionally an alkali metal or a quaternary ammonium salt thereof.

In certain example embodiments, the ω-carboxyalkanoate ester substituents are linear C₄ or higher ω-carboxyalkanoate ester substituents chosen from succinate mono-ester substituents, glutarate mono-ester substituents, any substituted derivatives thereof, and any combination thereof, optionally wherein the substituted derivatives thereof comprise alkanoate substituents, alkyl substituents, hydroxyalkyl substituents, or any combination thereof.

In certain example embodiments, the amphiphilic cellulose derivative comprises a 1/1 or greater molar ratio of ω-carboxyalkanoate ester substituents to available hydroxyl groups, and/or wherein the amphiphilic cellulose derivative comprises an average of 25% or greater ω-carboxyalkanoate ester substituents based on total substituents. In some embodiments, the amphiphilic cellulose derivative comprises an average of 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to/or 100% of ω-carboxyalkanoate ester substituents based on total substituents.

In certain example embodiments, the cellulose derivative comprises an average degree of substitution of available hydroxyl groups of 1.8 or greater. In some embodiments, the average degree of substitution of available hydroxyl groups is 1.8, 1.9, 2.0, 2.1, 2.2, 2.3, 2.4, 2.5, or greater.

In certain example embodiments, the cellulose derivative comprises ester substituents having the formula —C(═O)R, ether substituents having the formula —OR, or any combination thereof. In some embodiments, R is independently chosen at each occurrence from hydroxyl groups, alkyl ester substituents, alkoxide ester substituents, and any combination thereof. In some embodiments, R is independently chosen at each occurrence from —OH and an alkyl.

In certain example embodiments, wherein the cellulose derivative comprises acetate substituents, propionate substituents, butyrate substituents, hydroxyalkyl substituents, hydroxy functional polyether substituents, or any combination thereof.

In certain example embodiments, the cellulose derivative is cellulose acetate, cellulose propionate, cellulose acetate propionate, cellulose acetate butyrate, hydroxypropyl cellulose, hydroxyethyl cellulose, or any combination thereof.

In certain example embodiments, the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxypropyl cellulose, hydroxyethyl cellulose, or any combination thereof.

In certain example embodiments, the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

In certain example embodiments, the amphiphilic cellulose derivative comprises a number-average molecular weight (Mn) of 15,000 g/mol or greater. In certain example embodiments, the amphiphilic cellulose derivative comprises a number-average molecular weight (Mn) of 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol or greater.

In certain example embodiments, the amphiphilic cellulose derivative is soluble in an organic solvent, optionally chosen from an alcohol, tetrahydrofuran, chloroform, acetone, dimethyl sulfoxide, dimethylformamide, dimethylacetamide, and any combination thereof.

In certain example embodiments, the amphiphilic cellulose derivative is water soluble at 50 mg/mL or greater. In certain example embodiments, the amphiphilic cellulose derivative is water soluble at 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL.

In certain example embodiments, the amphiphilic cellulose derivative exhibits a surface tension of 35 to 70 mN/m. In certain example embodiments, the amphiphilic cellulose derivative exhibits a surface tension of 60 mN/m or less. In certain example embodiments, the amphiphilic cellulose derivative exhibits a surface tension of 50 mN/m or less. In certain example embodiments, the amphiphilic cellulose derivative exhibits a surface tension of 40 mN/m or less. In certain example embodiments, the amphiphilic cellulose derivative exhibits a surface tension of 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m, 61 mN/m, 62 mN/m, 63 mN/m, 64 mN/m, 65 mN/m, 66 mN/m, 67 mN/m, 68 mN/m, 69 mN/m, to/or 70 mN/m. In certain example embodiments, the amphiphilic cellulose derivative exhibits a surface tension of greater than 0 to 60 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m. In certain example embodiments, the amphiphilic cellulose derivative exhibits a surface tension of greater than 0 to 50 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m. In certain example embodiments, the amphiphilic cellulose derivative exhibits a surface tension of greater than 0 to 40 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m.

In certain example embodiments, the amphiphilic cellulose derivative is capable of increasing nucleation induction time of a supersaturated aqueous solution comprising the amphiphilic cellulose derivative and one or more agents as compared to a solution without the amphiphilic cellulose derivative. In some embodiments, the one or more agents are one or more poorly soluble agents.

In certain example embodiments, the nucleation induction time is increased about 2 to about 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the amphiphilic cellulose derivative exhibits a surface tension 50 mN/m or less, preferably 40 mN/m or less, and wherein the nucleation induction time is increased about 2 to 45 fold or 12 to 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold. In certain example embodiments, the nucleation induction time is increased about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the nucleation induction time of the supersaturated aqueous solution is greater than 0 minutes but less than 25 minutes or less than 10 minutes. In some embodiments, the nucleation induction time of the supersaturated aqueous solution is greater than 0 minutes to/or 1, 2, 3, 4, 5, 6, 7, 8, 9, 10, 11, 12, 13, 14, 15, 16, 17, 18, 19, 20, 21, 22, 23, 24 up to 25 minutes. In some embodiments, the nucleation induction time of the supersaturated aqueous solution is greater than 0 minutes to/or 1, 2, 3, 4, 5, 6, 7, 8, 9, up to 10 minutes.

In certain example embodiments, such amphiphilic cellulose derivatives are prepared by any of the methods of the present disclosure. In certain example embodiments, amphiphilic cellulose derivatives are used to prepare amorphous solid dispersions of the present disclosure.

### Amorphous Solid Dispersions and Formulations Thereof

Described in several exemplary embodiments herein are amorphous solid dispersions that can contain the cellulose derivatives, such as amphiphilic cellulose derivatives, of the present disclosure. Such cellulose derivatives can be prepared by any of the methods of the present disclosure. The ASDs can contain the cellulose derivatives, such as ω-carboxyalkanoate derivatives, with e.g., poorly soluble drugs and/or other active species. Without being bound by theory, such ASDs can provide unexpectedly long duration of supersaturation of those drugs in aqueous solution, due to the unexpectedly excellent ability of these cellulose derivatives, e.g., ω-carboxyalkanoate derivatives, to stabilize these supersaturated solutions against drug crystallization, thereby strongly enhancing bioavailability in the case of orally administered drugs. The ASDs can be prepared into various dosage forms, such as oral dosage forms, for administration to a subject.

Described in exemplary embodiments herein are amorphous solid dispersion comprising a cellulose ester or a cellulose derivative comprising one or more ω-carboxyalkanoate ester substituents; and one or more agents, optionally wherein one or more of the one or more agents is poorly water soluble.

In certain example embodiments, the one or more agents are crystalline organic molecule(s). In certain example embodiments, the one or more agents are each independently selected from a weakly basic pharmaceutical compound, a neutral pharmaceutical compound, or a weakly acidic pharmaceutical compound. In certain example embodiments, one or more of the one or more agents is a therapeutic agent. In certain example embodiments, one or more of the one or more agents is an immunomodulator (including but not limited to an immunosuppressant), an antipyretic, an anxiolytic, an antipsychotic, an anticonvulsant, an analgesic, an antispasmodic, an anti-inflammatory, an antihistamine, an anti-infective (including but not limited to an anti-bacterial agent, an anti-fungal agent, or an anti-viral agent), a chemotherapeutic, a vasomodulator, an anti-diabetic, a radiation sensitizer, a chemotherapeutic sensitizer, or any combination thereof. In certain example embodiments, the one or more agents are each independently selected from nonsteroidal anti-inflammatory agents (NSAIDs), antifungal agents, chemotherapeutics, or any combination thereof. In certain example embodiments, the one or more agents are each independently selected from Celecoxib, Enzalutamide, Posaconazole, or any combination thereof.

In certain example embodiments, the one or more agents, optionally one or more of the poorly water soluble agents, has increased bioavailability as compared to its free form. In certain example embodiments, the one or more agents, optionally one or more of the poorly soluble agents, has increased oral bioavailability as compared to its free form.

In certain example embodiments, the amorphous solid dispersion is prepared by a method comprising reacting a cellulose or cellulose derivative and an aliphatic cyclic anhydride, wherein reacting results in the ω-carboxyalkanoate ester substituent of the cellulose derivative. In certain example embodiments, the amorphous solid dispersion is prepared by a method comprising reacting a cellulose or cellulose derivative and one or more aliphatic cyclic anhydrides, wherein reacting results in the ω-carboxyalkanoate ester substituent of the cellulose derivative. In some embodiments two or more anhydrides are mixed or contained in a single species. For example, the cellulose or cellulose ester can be reacted with succinic and glutaric anhydrides together.

In certain example embodiments, the aliphatic cyclic anhydride is a succinic anhydride, glutaric anhydride, or a substituted derivative thereof, optionally wherein the derivative thereof comprises an alkanoate substituent, an alkyl substituent, a hydroxyalkyl substituent, or any combination thereof.

In certain example embodiments, reacting comprises a ring opening reaction.

In certain example embodiments, the cellulose or cellulose derivative is a cellulose alkanoate, an alkyl cellulose ether, a hydroxyalkyl cellulose ether, a cellulose acetate propionate, a cellulose acetate butyrate, a cellulose acetate, or any combination thereof.

In certain example embodiments, the cellulose derivative comprises ester substituents having the formula —C(═O)R, ether substituents having the formula —OR, or combinations thereof. In some embodiments, R is independently chosen from hydroxyl groups, alkyl ester substituents, and alkoxide ester substituents. In certain example embodiments, the cellulose derivative comprises ester substituents having the formula —C(═O)R, ether substituents having the formula —OR, or any combination thereof, optionally wherein R is independently chosen at each occurrence from —OH and an alkyl.

In certain example embodiments, the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxyethyl cellulose, or any combination thereof. In certain example embodiments, the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

In certain example embodiments, the method of preparing the amorphous solid dispersion eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both.

In certain example embodiments, one or more side reactions are eliminated, reduced, minimized, or any combination thereof by optimizing one or more reaction parameters, optionally wherein the one or more reaction parameters is reaction time, reaction temperature, stoichiometry, solvent, product isolation, product isolation, or any combination thereof.

In certain example embodiments, the amorphous solid dispersion is prepared by a method comprising dissolving the one or more agents, optionally one or more poorly water soluble agents, and one or more amphiphilic cellulose derivatives in one or more organic solvent(s), optionally chosen from an alcohol, tetrahydrofuran, chloroform, acetone, dimethyl sulfoxide, dimethylformamide, and dimethylacetamide; removing the solvent from one or more agents and the one or more amphiphilic cellulose derivatives, thus forming the amorphous solid dispersion.

In certain example embodiments, one or more of the one or more amphiphilic cellulose derivatives comprises one or more ω-carboxyalkanoate ester substituents.

In certain example embodiments, the amphiphilic cellulose derivative comprises ester substituents having the formula —C(═O)R, ether substituents having the formula —OR, or combinations thereof. In some embodiments, R is independently chosen at each occurrence from hydroxyl groups, alkyl ester substituents, alkoxide ester substituents, and any combination thereof. In some embodiments, R is independently chosen at each occurrence from —OH and an alkyl.

In certain example embodiments, the amphiphilic cellulose derivative comprises acetate substituents, propionate substituents, butyrate substituents, hydroxy functional polyether substituents, or combinations thereof.

In certain example embodiments, the amphiphilic cellulose derivative is MCC succinate, CA320S Succinate (low DS), CA320S (high DS), CA320S glutarate, CA398 Succinate, Cellulose Acetate Butyrate (CAB-553-0.4) Succinate, or Cellulose Acetate Propionate (CAP-504-0.2) Glutarate. In certain example embodiments, the amphiphilic cellulose derivative is MCC succinate, CA320S succinate (low DS), CA320S (high DS), CA320S glutarate, CA398 succinate, cellulose acetate butyrate (CAB-553-0.4) succinate, cellulose acetate propionate (CAP-504-0.2) glutarate, or cellulose acetate glutarate succinate

In certain example embodiments, the amphiphilic cellulose derivative comprises a number-average molecular weight (Mₙ) of 15,000 g/mol or greater. In certain example embodiments, the amphiphilic cellulose derivative comprises a number-average molecular weight (Mn) of 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol or greater.

In certain example embodiments, the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

In certain example embodiments, the amphiphilic cellulose derivative is water soluble at 50 mg/mL or greater. In certain example embodiments, the amphiphilic cellulose derivative is water soluble at 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL.

In certain example embodiments, the amphiphilic cellulose derivative exhibits a surface tension of 35 to 70 mN/m. In certain example embodiments, the amphiphilic cellulose derivative exhibits a surface tension of 60 mN/m or less. In certain example embodiments, the amphiphilic cellulose derivative exhibits a surface tension of 50 mN/m or less. In certain example embodiments, the amphiphilic cellulose derivative exhibits a surface tension of 40 mN/m or less. In certain example embodiments, the amphiphilic cellulose derivative exhibits a surface tension of 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m, 61 mN/m, 62 mN/m, 63 mN/m, 64 mN/m, 65 mN/m, 66 mN/m, 67 mN/m, 68 mN/m, 69 mN/m, to/or 70 mN/m. In certain example embodiments, the amphiphilic cellulose derivative exhibits a surface tension of greater than 0 to 60 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m, 51 mN/m, 52 mN/m, 53 mN/m, 54 mN/m, 55 mN/m, 56 mN/m, 57 mN/m, 58 mN/m, 59 mN/m, 60 mN/m. In certain example embodiments, the amphiphilic cellulose derivative exhibits a surface tension of greater than 0 to 50 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m, 41 mN/m, 42 mN/m, 43 mN/m, 44 mN/m, 45 mN/m, 46 mN/m, 47 mN/m, 48 mN/m, 49 mN/m, 50 mN/m. In certain example embodiments, the amphiphilic cellulose derivative exhibits a surface tension of greater than 0 to 40 mN/m, such as greater than 0 to/or 1 mN/m, 2 mN/m, 3 mN/m, 4 mN/m, 5 mN/m, 6 mN/m, 7 mN/m, 8 mN/m, 9 mN/m, 10 mN/m, 11 mN/m, 12 mN/m, 13 mN/m, 14 mN/m, 15 mN/m, 16 mN/m, 17 mN/m, 18 mN/m, 19 mN/m, 20 mN/m, 21 mN/m, 22 mN/m, 23 mN/m, 24 mN/m, 25 mN/m, 26 mN/m, 27 mN/m, 28 mN/m, 29 mN/m, 30 mN/m, 31 mN/m, 32 mN/m, 33 mN/m, 34 mN/m, 35 mN/m, 36 mN/m, 37 mN/m, 38 mN/m, 39 mN/m, 40 mN/m.

In certain example embodiments, the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the supersaturated aqueous solution of the one or more agents and a carboxylic acid functional cellulose derivative comprises loading ratio of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

In certain example embodiments, the nucleation induction time of a supersaturated aqueous solution of the one or more agents is greater than 0 minutes but less than 25 minutes or less than 10 minutes.

In certain example embodiments, the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold. In certain example embodiments, the nucleation induction time is increased about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold. In certain example embodiments, the nucleation induction time is increased about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or 45 fold.

In certain example embodiments, the one or more amphiphilic cellulose derivatives are prepared by a method comprising mono-esterification of a plurality of diacids or derivatives, with a plurality of hydroxyl groups of a cellulose or a cellulose derivative.

In certain example embodiments, each of the plurality of diacids are independently dicarboxylic acids. In some embodiments, the each of the plurality of diacids are independently α,ω-dicarboxylic acids. In some embodiments, each of the plurality of diacids are independently C₄, C₅ or higher α,ω-dicarboxylic acids. In some embodiments, each of the plurality of diacids are independently a C₄ or C₅ α,ω-dicarboxylic acid.

In certain example embodiments, the dicarboxylic acids are each independently selected from succinic acid or glutaric acid.

In certain example embodiments, the plurality of diacid derivatives are anhydrides. In some embodiments, the anhydrides are independently carboxylic anhydrides. In some embodiments, the carboxylic anhydrides are linear or cyclic anhydrides. In some embodiments, the carboxylic anhydrides are C₄-C₈ or higher linear or cyclic anhydrides. In some embodiments, the carboxylic anhydrides are C₄ or C₅ linear or cyclic anhydrides.

In certain example embodiments, the one or more agents and the one or more amphiphilic cellulose derivatives are loaded in a 1:99 to 50:50 wt./wt. ratio or greater. In certain example embodiments, the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

### Pharmaceutical Formulations

Described in certain example embodiments herein are pharmaceutical formulations that can contain an amorphous solid dispersion of as described in greater detail elsewhere herein. In some embodiments, a primary or secondary active agent, which can be contained in the ASD of the present disclosure, can be provided in the pharmaceutically acceptable salt form of the active agents(s). Suitable pharmaceutically acceptable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate. In some embodiments, the one or more ω-carboxyalkanoate ester substituents are in a salt form. In some embodiments, the one or more ω-carboxyalkanoate ester substituents are in a monovalent salt form. In some embodiments, one or more ω-carboxyalkanoate ester substituents are present as alkali metal cation or tetraalkylammonium cation.

In certain example embodiments, the one or more agents, optionally one or more poorly soluble agents, has increased oral bioavailability as compared to the free form of the one or more agents. In certain example embodiments, the amorphous solid dispersion is in the dosage form of granulated particles or a tablet. Other dosage forms are described elsewhere herein.

### Pharmaceutically Acceptable Carriers and Secondary Ingredients and Agents

The pharmaceutical formulation can include, where appropriate, a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation also includes an amount, such as an effective amount, of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent contained in the pharmaceutical formulation and/or included in the ASD preparation of the present disclosure, is an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In some embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when present in the formulation, is included at an amount from any value greater than 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents in the pharmaceutical formulation. In additional embodiments, the effective amount of the secondary active agent, when present in the formulation, is included at an amount from any value greater than 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et, al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

In some embodiments, the dosage form is suitable for topical administration, such as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. Dosage forms adapted for topical administration in the mouth include lozenges, pastilles, and mouth washes.

In some embodiments, the dosage form is suitable for nasal or inhalation administration. Such dosage forms include, without limitation, aerosols, solutions, suspension drops, gels, or dry powders. In some embodiments, the ASD is prepared in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of the ASD of the present disclosure and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension an ASD of the present disclosure. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

In some embodiments, the dosage form is adapted for vaginal or rectal administration. Dosage forms adapted for vaginal or rectal administration can be presented as pessaries, tampons, creams, gels, pastes, foams, powders, or spray formulations containing the ASD. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses, such as ASDs of the present disclosure, can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s), such as those containing the ASDs of the present disclosure, described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof. In some embodiments, the co-therapy (e.g., a secondary active agent) can be included in the ASD preparation along with the primary active agent, such as a poorly soluble active agent.

In some embodiments, the co-therapy or combination therapy can additionally include but are not limited to, polynucleotides, amino acids, peptides, polypeptides, antibodies, aptamers, ribozymes, hormones, immunomodulators, (including but not limited to an immunosuppressant) antipyretics, anxiolytics, antipsychotics, analgesics, antispasmodics, anti-inflammatories, anti-histamines, anti-infectives (including but not limited to an anti-bacterial, an anti-viral, or an anti-fungal), chemotherapeutics, and any combination thereof.

### Administration of the Pharmaceutical Formulations

The pharmaceutical formulations and/or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, and/or formulations, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, and/or formulations, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

Described in several exemplary embodiments herein are methods of using the compounds, compositions, and/or formulations described herein. In some embodiments, they can be administering to a subject, such as a subject in need of treatment or prevention of a disease, disorder, condition or symptom thereof. In some embodiments, a method includes administering to a subject a formulation comprising a cellulose ester as in any one of the preceding claims; a formulation comprising a cellulose derivative comprising one or more ω-carboxyalkanoate ester substituents as described herein; an amorphous solid dispersion as described herein; a pharmaceutical formulation as described herein; or any combination thereof.

In some embodiments, the formulation, amorphous solid dispersion, pharmaceutical formulation, or any combination thereof is effective to treat a disease, condition, disorder, or a symptom thereof in the subject. Non-limiting, exemplary diseases, conditions and disorders include, cancer, autoimmune diseases, infectious diseases, genetic diseases and disorders, idiopathic diseases and disorders, pain, inflammatory diseases, neuropathic diseases, cardiovascular diseases, musculoskeletal diseases, nervous system diseases, renal diseases, gastrointestinal diseases, lung diseases, liver diseases, ocular diseases, auditory diseases, olfactory diseases, and/or the like.

### METHODS OF USE

Described in several exemplary embodiments herein are methods of using the compounds, compositions, and/or formulations described herein, such as for preparing a pharmaceutical formulation and/or dosage forms and delivery to a subject in need thereof. In some embodiments, methods can include administering the compounds, compositions, and/or formulations described herein to a subject, such as a subject in need of treatment or prevention of a disease, disorder, condition or symptom thereof. Without being bound by theory, the compounds, compositions, and/or formulations described herein can provide increased bioavailability and/or delivery of a therapeutic agent, such as one that is poorly water soluble, to the subject. In some embodiments, a method includes administering to a subject a formulation comprising a cellulose ester or derivative thereof of the present disclosure. In some embodiments, the method includes administering a formulation comprising a cellulose derivative comprising one or more ω-carboxyalkanoate ester substituents described in greater detail elsewhere herein; an amorphous solid dispersion described in greater detail elsewhere herein, a pharmaceutical formulation as described in greater detail elsewhere herein, or any combination thereof. In some embodiments, the one or more ω-carboxyalkanoate ester substituents are in a salt form. In some embodiments, the one or more ω-carboxyalkanoate ester substituents are in a monovalent salt form. In some embodiments, one or more ω-carboxyalkanoate ester substituents are present as alkali metal cation or tetraalkylammonium cation.

The compounds, compositions, and/or formulations (such as ASD preparations of the present disclosure) and/or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

In some embodiments, the compounds, compositions, and/or formulations (such as ASD preparations of the present disclosure) and/or dosage forms thereof described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof. In some embodiments, the co-therapy (e.g., a secondary active agent) can be included in the ASD preparation along with the primary active agent, such as a poorly soluble active agent.

In some embodiments, the co-therapy or combination therapy can additionally include but are not limited to, polynucleotides, amino acids, peptides, polypeptides, antibodies, aptamers, ribozymes, hormones, immunomodulators (including but not limited to an immunosuppressant), antipyretics, anxiolytics, antipsychotics, analgesics, antispasmodics, anti-inflammatories, anti-histamines, anti-infectives (including but not limited to an antibacterial, an antiviral, or an antifungal), chemotherapeutics, and combinations thereof.

In some embodiments, the formulation, amorphous solid dispersion, pharmaceutical formulation, or any combination thereof is effective to treat a disease, condition, disorder, or a symptom thereof in the subject.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C., and 1 atmosphere.

### Example 1—Exemplary Cellulose Ester Derivatives for ASD Applications

A general workflow for evaluating cellulose ester derivatives for ASD applications can include evaluating polymer solubility, nucleation time, and surface tension. Polymer solubility was evaluated by examining solubility in pH 6.8 buffer and organic solvents. Nucleation induction time was measured using 9 different polymers and 3 model therapeutic compounds. Surface tension was determined by correlating surface tension and induction time. Tables 2-3 below shows polymers evaluated in vitro. Table 4 shows the physiochemical information on the polymer substrates. Table 5 shows the organic and aqueous solubility of the polymer evaluated in vitro. Table 6 shows the glass transition temperature of the polymers evaluated in vitro.

TABLE 2
Composition Data of Polymers Used for in vitro evaluation

| | Starting | Starting | Starting | | Starting | | | |
|---|---|---|---|---|---|---|---|---|
| Starting | Polymer | Polymer | Polymer | Starting | Polymer | | Product | |
| polymer | DS | DS | DS | Polymer | Available | Polymer | DS ω- | |
| substrate | Acetyl | Propionyl | Butyryl | total DS | DS (OH) | Product | carboxyl | Abbreviation |
| MCC (1) | 0 | 0 | 0 | 0 | 3 | MCC | 2.90 | Cell-succ |
| | | | | | | Succinate | | |
| CA320S | 1.82 | 0 | 0 | 1.82 | 1.18 | CA320S | 0.75 | CA320succlow |
| (4) | | | | | | Succinate | | |
| | | | | | | (low DS) | | |
| CA320S | 1.82 | 0 | 0 | 1.82 | 1.18 | CA320S | 1.26 | CA320succhigh |
| (4) | | | | | | Succinate | | |
| | | | | | | (high DS) | | |
| CA320S | 1.82 | 0 | 0 | 1.82 | 1.18 | CA320S | 1.33 | CA320GA |
| (4) | | | | | | Glutarate | | |
| CA398 | 2.47 | 0 | 0 | 2.47 | 0.53 | CA398 | 0.58 | CA398succ |
| (2) | | | | | | Succinate | | |
| CAB- | 0.14 | 0 | 1.99 | 2.13 | 0.87 | Cellulose | 0.58 | CABsucc |
| 553-0.4 | | | | | | Acetate | | |
| (3) | | | | | | Butyrate | | |
| | | | | | | (CAB- | | |
| | | | | | | 553-0.4) | | |
| | | | | | | Succinate | | |
| CAB- | 0.14 | 0 | 1.99 | 2.13 | 0.87 | Cellulose | 0.91 | CABGA |
| 553-0.4 | | | | | | Acetate | | |
| (3) | | | | | | Butyrate | | |
| | | | | | | (CAB- | | |
| | | | | | | 553-0.4) | | |
| | | | | | | Glutarate | | |
| CAP- | 0.04 | 2.09 | 0 | 2.13 | 0.87 | Cellulose | 0.51 | CAPsucc |
| 504-0.2 | | | | | | Acetate | | |
| (5) | | | | | | Propionate | | |
| | | | | | | (CAP- | | |
| | | | | | | 504.02) | | |
| | | | | | | Succinate | | |
| CAP- | 0.04 | 2.09 | 0 | 2.13 | 0.87 | Cellulose | 0.93 | CAPGA |
| 504-0.2 | | | | | | Acetate | | |
| (5) | | | | | | Propionate | | |
| | | | | | | (CAP- | | |
| | | | | | | 504-0.2) | | |
| | | | | | | Glutarate | | |

TABLE 3

| | | Cellulose- | | |
|---|---|---|---|---|
| Cellulose | | based | Product | |
| Substrate | Structure (compound no.) | product | DS (SA) | Abbreviation |
| Microcrystalline cellulose (MCC) | 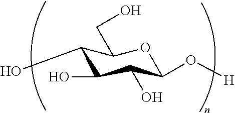 | MCC succinate | 2.9 | Cell-succ |
| | (1) | | | |
| Cellulose Acetate 398 (CA398) | 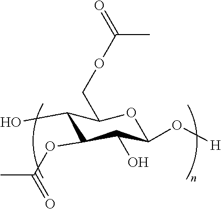 | CA-398-30 Succinate | 0.58 | CA398succ |
| | (2) | | | |
| Cellulose Acetate Butyrate (CAB) | 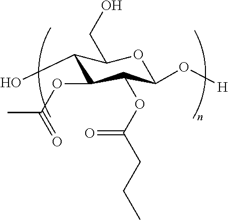 | CAB-553- 0.4 Succinate | 0.58 | CABsucc |
| | (3) | | | |
| Cellulose Acetate 320S (CA320S) | 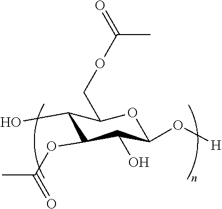 | CA320S Succinate | 1.26 | CA320succhigh |
| | (2) | | | |
| Cellulose Acetate Propionate (CAP) | 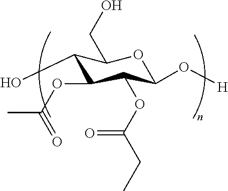 | CAP-504- 0.2 Succinate | 0.51 | CAPsucc |
| | (4) | | | |
| Hydroxyethyl cellulose | 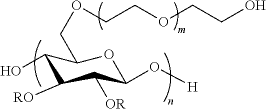 | HEC Succinate | 1.83 | HECsucc |
| | (5) | | | |
| | R = | | | |
| | H₂(CH₂CH₂O)ₘCH₂CH₂OH | | | |
| | | Cellulose- | | |
| Cellulose | | based | Product | |
| Substrate | Structure (compound no.) | product | DS (GA) | Abbreviation |
| Cellulose Acetate 398 (CA398) | 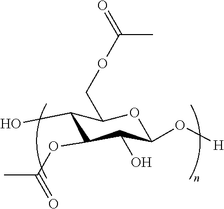 | CA-398-30 Glutarate | 0.42/0.53 | CA398GA |
| | (2) | | | |
| Cellulose Acetate Butyrate (CAB) | 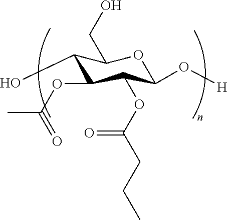 | CAB-553- 0.4 Glutarate | 0.91/0.87 | CABGA |
| | (3) | | | |
| Cellulose Acetate 320S (CA320S) | 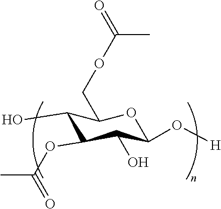 | CA 320S Glutarate | 1.33/1.18 | CA320GA |
| | (2) | | | |
| Cellulose Acetate Propionate CAP) | 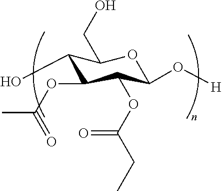 | CAP-504- 0.2 Glutarate | 0.93/0.87 | CAPGA |
| | (4) | | | |
| Hydroxyethyl cellulose | 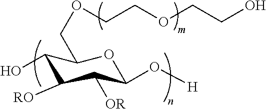 | | | HECGA |
| | (5) | | | |
| | R = | | | |
| | H₂(CH₂CH₂O)ₘCH₂CH₂OH | | | |

NOTE: Structures are depicted as above only for simplicity and clarity, and are not intended to imply regioselective substitution.

TABLE 4
below shows physicochemical information on the polymer substrates.
Table 4. Physiochemical information on polymer substrates

| | | | | | | | Available |
|---|---|---|---|---|---|---|---|
| | Sub- | Sub- | | | | | DS |
| | strate | strate | | | | | (OH) for |
| Starting | Mn | T_{g} | DS | DS | DS | Total | esterfi- |
| Substrate | (10*3) | (° C.) | (Ac)* | (Pr)* | (Bu)* | DS | cation |
| MCC | ~36 | N/A | 0 | 0 | 0 | 0 | 3 |
| CA | 20 | 180 | 1.82 | 0 | 0 | 1.82 | 1.18 |
| 320S | | | | | | | |
| CA- | 50 | 189 | 2.47 | 0 | 0 | 2.47 | 0.53 |
| 398-30 | | | | | | | |
| CAP- | 15 | 159 | 0.04 | 2.09 | 0 | 2.13 | 0.87 |
| 504-0.2 | | | | | | | |
| CAB- | 20 | 136 | 0.14 | 0 | 1.99 | 2.13 | 0.87 |
| 553-0.4 | | | | | | | |

Ac = acetyl,
Pr = propionyl,
Bu = butyryl

For polymer solubility evaluation, insolubility was determined at 2 mg/mL by visual inspection. All polymers are soluble at lower concentrations (e.g., 50 μg/mL, polymer concentration in nucleation induction time experiments). All polymers are soluble in at least one low b.p. organic solvent (e.g., MeOH, EtOH, THF, CHCl₃, and acetone) and polar aprotic organic solvents (e.g., DMSO, DMF, and DMAc). Solubility results are shown Table 5 below.

TABLE 5
Polymer Solubility

| | Cell- | | | | | | | | |
|---|---|---|---|---|---|---|---|---|---|
| | succ | CA320succhighDS | CA320succlowDS | CA320GA | CA398succ | CABSucc | CABGA | CAPsucc | CAPGA |
| pH 6.8 | √ | √ | √ | √ | X | X | X | X | X |
| buffer | | | | | | | | | |
| Solubility | 31.4 | 10-20 | 10-20 | >20 | N.A. | N.A. | N.A. | N.A. | N.A. |
| (mg/mL) | | | | | | | | | |

For evaluation of the thermal properties of the polymers, dynamic scanning calorimetry (DSC) was performed using a TA Instruments Q2000 DSC instrument. Data is shown in Table 6.

Table 6 shows the glass transition temperature data of the polymers evaluated in the in vitro experiments.

TABLE 6

| Polymer | Tg (° C.) |
|---|---|
| Cell-succ | 175 |
| CA320Ssucclow | 159 |
| CA320Ssucchigh | 170 |
| CA398succ | 143 |
| CAPsucc | 141 |
| CABsucc | 113 |
| CAPGA | 129 |
| CA320SGA | 159 |
| CABGA | 108 |

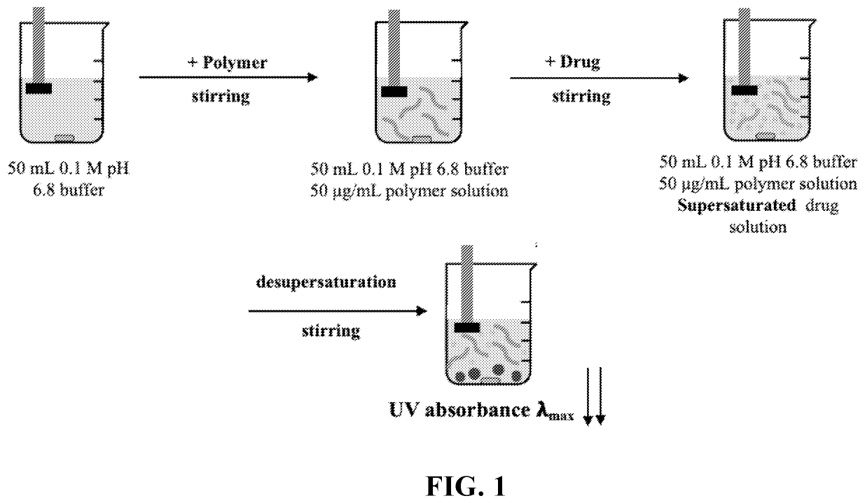

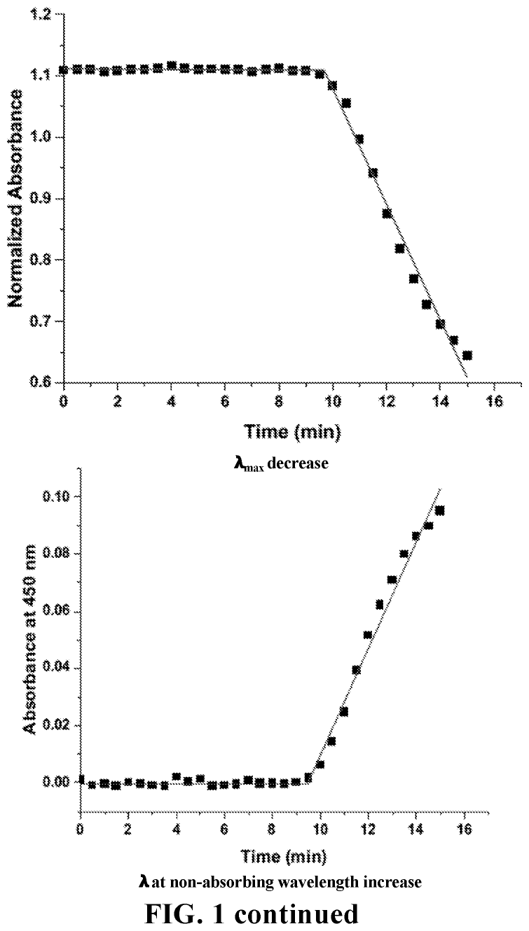

FIG. 1 shows the experimental methodology for evaluating nucleation induction time. Nucleation can be evaluated by monitoring the λmax and λ at non-absorbing wavelength over time.

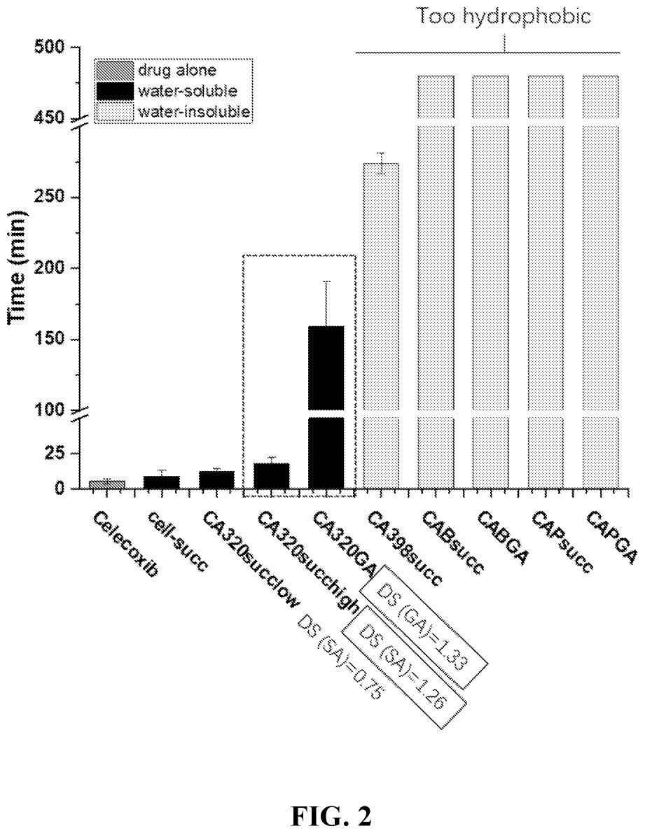

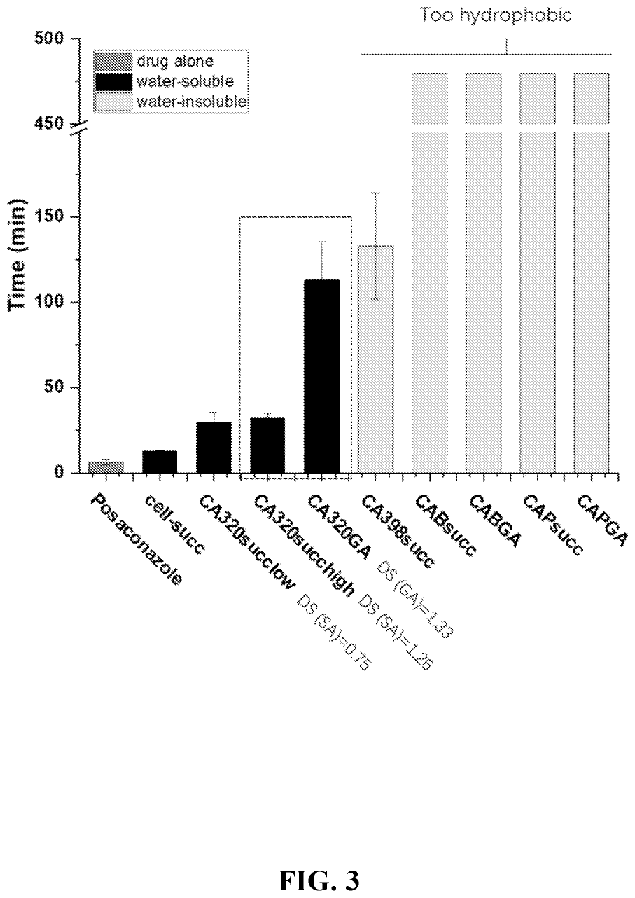

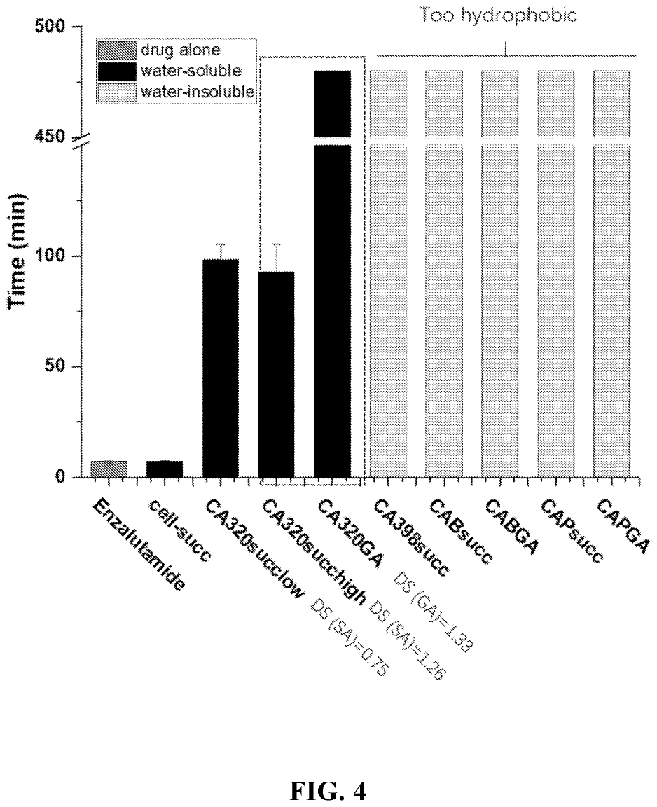

Three model therapeutic agents were evaluated: celecoxib, posaconazole, and enzalutamide. Celecoxib is a weak acidic compound that is unionized at pH 6.8. It is a nonsteroidal antiinflammatory drug (NSAID). Posaconazole is a weak basic compound that is unionized at a pH 6.8. Posaconazole is used to treat fungal infections. Enzalutamide is a neutral compound that is clinically used to treat prostate cancer. FIGS. 2-4 show the nucleation induction time results for each of the model drugs. For celecoxib, CA320succhigh and CA320GA had similar Ac and COOH DS (degree of substitution), the only difference is the number of carbons in the tether linkage.

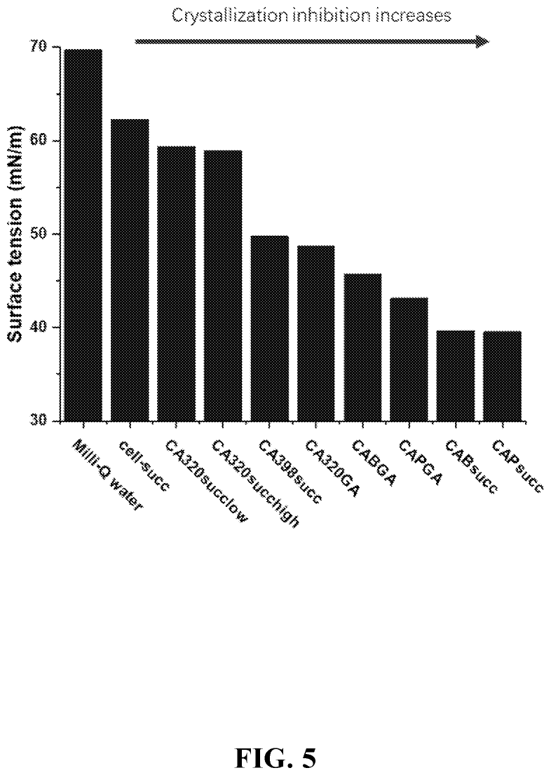

FIG. 5 shows the relationship between surface tension and nucleation induction time for the tested polymers. Surface tension reflects the amphiphilicity of a polymer. The nucleation induction time results were as follows: cell-succ<CA320succ high≈CA320succlow<CA320GA<CA398succ<CABGA≈CAPGA≈CABsucc≈CAPsucc. A general trend was observed with the polymers evaluated. Better crystallization inhibition performance was observed to be related to lower surface tension.

It was observed that water-insoluble cellulose ester succinates and glutarates exhibit excellent crystallization inhibition properties, while most water-soluble cellulose ester succinates and glutarates do not inhibit drug crystallization to an effective extent. CA320GA is a particularly promising candidate for ASD applications as it is water-soluble and also was observed to have good crystallization inhibition. Polymer solutions with lower surface tension usually exhibit better performance in preventing drug crystallization.

### Example 2—Exemplary Reaction Methods

### Cellulose Acetate (CA 320S) Glutarate

Reaction of Cellulose Acetate (CA 320S) with Commercial Glutaric Anhydride

CA 320S (2 g, 8.38 mmol) was pre-dried at 50° C. in a vacuum oven overnight and then dissolved in 200 ml of anhydrous 1,3-dimethyl-2-imidazolidinone (DMI) for 5 hours at 50° C. The solution was turned off and continued to dissolve overnight at room temperature under nitrogen. 0.2 g (1.64 mmol) of 4-dimethylaminopyridine (DMAP) was dissolved in 6 mL of anhydrous pyridine then added dropwise to the reaction solution at room temperature under nitrogen. The temperature was increased to 80° C. Glutaric anhydride (1.15 g, 1 equiv.) was dissolved in 5mls of anhydrous DMI and added to the reaction solution dropwise at 80° C. under nitrogen. The reaction was run for 72 h. After 72 h, the mixture was slowly poured into vigorously stirring MeOH to reduce the viscosity of the reaction solution. Then, the reaction solution was slowly precipitated in 0.1N HCl. The polymer product was vacuum filtered and washed with diH₂O. The polymer was transferred to dialysis tubing where it continued to dialyze against diH₂O for several days prior to freeze drying.

### Cellulose Acetate Butyrate (CAB-553-0.4) Succinate

Reaction of Cellulose Acetate Butyrate (CAB-553-0.4) with Commercial Succinic Anhydride

CAB-553-0.4 (4 g, 13.04 mmol) was dissolved in 140 mL of anhydrous DMI for 1 h at room temperature under nitrogen and mechanical stirring. DMAP (0.4 g, 3.27 mmol) was dissolved in 12 mL of anhydrous pyridine and added dropwise to the reaction mixture at room temperature. Then, the temperature was increased to 90° C. Succinic anhydride (5.11 g, 4.5 equiv.) was dissolved in 15 mL DMI and added dropwise at 90° C. The reaction was run for 48 h. Then, the reaction was precipitated in diH₂O, resolubilized in acetone, and reprecipitated in 0.1N HCl. The product was transferred to dialysis tubing and dialyzed against diH₂O for several days prior to freeze-drying.

### Cellulose Acetate Propionate (CAP-504-0.2) Glutarate

Reaction of Cellulose Acetate Propionate (CAP-504-0.2) with Commercial Glutaric Anhydride

CAP-504-0.2 (4 g, 14.24 mmol) was pre-dried in a vacuum oven at 50° C. overnight then dissolved in 120 mL anhydrous DMI overnight at room temperature. DMAP (0.3 g, 2.45 mmol) was dissolved in 12 mL anhydrous pyridine and added dropwise to the reaction solution at room temperature under nitrogen. Then, the reaction was heated to 80° C. Glutaric anhydride (4.21 g, 3 equiv.) was dissolved in 9 mL DMI and added dropwise at 80° C. under nitrogen atmosphere. The reaction was run for 4 days. The reaction was precipitated in diH₂O, vacuum filtered, dialyzed against diH₂O, and freeze dried.

### MCC (Cellulose) Succinate and Glutarate (Mixed Ring Synthesis)

Reaction of Cellulose in DMAc/LiCl Solution with Commercial Succinic Anhydride and Glutaric Anhydride

MCC (1 g, 6.16 mmol) was dissolved in a 50 mL anhydrous N, N-dimethylacetamide (DMAc)/LiCl solution overnight according to a previous literature protocol. After dissolution was complete, DMAP (0.2 g, 1.64 mmol) was dissolved in 3 mL anhydrous pyridine and added dropwise under nitrogen and mechanical stirring at room temperature. Then, the reaction was heated to 80° C. 2.5 g (1.2 equiv.) of glutaric anhydride and 1.50 g (0.8 equiv.) of succinic anhydride were dissolved in 7 mL of DMAc. At 80° C., the glutaric anhydride and succinic anhydride solution was added dropwise to the reaction solution under nitrogen. The reaction was run for 24 h. Then, the reaction was precipitated in 200 proof ethanol and vacuum filtered. Then, the product was transferred to dialysis tubing to dialyze against diH₂O. After several days, the product was protonated with 0.1N HCl, dialyzed against diH₂O for 24 h to remove any remaining acid, and freeze dried.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An amphiphilic cellulose derivative comprising:
a cellulose or a cellulose derivative, wherein one or more hydroxyl groups of the cellulose or the cellulose derivative are replaced by α,ω-dicarboxylic acid mono-ester (“ω-carboxyalkanoate ester”) substituents, optionally C₄ or higher ω-carboxyalkanoate ester substituents, any substituted derivatives thereof, or any combination thereof, and wherein the cellulose or the cellulose derivative is not crosslinked.

2. The amphiphilic cellulose derivative of aspect 1, wherein the amphiphilic cellulose derivative does not comprise oligomeric anhydrides, poly(anhydrides), side chains thereof, or any combination thereof, optionally crosslinked or capable of forming crosslinks between hydroxyl groups of different molecules of the amphiphilic cellulose derivative.

3. The amphiphilic cellulose derivative of aspects 1 or 2, wherein the ω-carboxyalkanoate ester substituents comprise a terminal carboxylic acid group or a monovalent salt thereof, optionally an alkali metal or a quaternary ammonium salt thereof.

4. The amphiphilic cellulose derivative of any one of aspects 1-3 wherein the ω-carboxyalkanoate ester substituents are linear C₄ or higher ω-carboxyalkanoate ester substituents chosen from succinate mono-ester substituents, glutarate mono-ester substituents, any substituted derivatives thereof, and any combination thereof, optionally wherein the substituted derivatives thereof comprise alkanoate substituents, alkyl substituents, hydroxyalkyl substituents, or any combination thereof.

5. The amphiphilic cellulose derivative of any one of aspects 1-4, wherein the amphiphilic cellulose derivative comprises a 1/1 or greater molar ratio of ω-carboxyalkanoate ester substituents to available hydroxyl groups, and/or wherein the amphiphilic cellulose derivative comprises an average of 25% or greater ω-carboxyalkanoate ester substituents based on total substituents.

6. The amphiphilic cellulose derivative of any one of aspects 1-5, wherein the cellulose derivative comprises an average degree of substitution of available hydroxyl groups of 1.8 or greater.

7. The amphiphilic cellulose derivative of any one of aspects 1-6, wherein the cellulose derivative comprises ester substituents having the formula —C(═O)R, ether substituents having the formula —OR, or any combination thereof, optionally wherein R is independently chosen at each occurrence from hydroxyl groups, alkyl ester substituents, alkoxide ester substituents, and any combination thereof, or optionally wherein R is independently chosen at each occurrence from —OH or an alkyl.

8. The amphiphilic cellulose derivative of any one of aspects 1-7, wherein the cellulose derivative comprises acetate substituents, propionate substituents, butyrate substituents, hydroxyalkyl substituents, hydroxy functional polyether substituents, or any combination thereof.

9. The amphiphilic cellulose derivative of any one of aspects 1-8, wherein the cellulose derivative is cellulose acetate, cellulose propionate, cellulose acetate propionate, cellulose acetate butyrate, hydroxypropyl cellulose, hydroxyethyl cellulose, or any combination thereof.

10. The amphiphilic cellulose derivative of any one of aspects 1-9, wherein the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxypropyl cellulose, hydroxyethyl cellulose, or any combination thereof.

11. The amphiphilic cellulose derivative of any one of aspects 1-10, wherein the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate.

12. The amphiphilic cellulose derivative of any one of aspects 1-11, wherein the amphiphilic cellulose derivative comprises a number-average molecular weight (Mn) of 15,000 g/mol or greater.

13. The amphiphilic cellulose derivative of any one of aspects 1-12, wherein the amphiphilic cellulose derivative is soluble in an organic solvent, optionally chosen from an alcohol, tetrahydrofuran, chloroform, acetone, dimethyl sulfoxide, dimethylformamide, dimethylacetamide, and any combination thereof.

14. The amphiphilic cellulose derivative of any one of the aspects 1-13, wherein the amphiphilic cellulose derivative is water soluble at 50 mg/mL or greater.

15. The amphiphilic cellulose derivative of any one of aspects 1-14, wherein the amphiphilic cellulose derivative exhibits a surface tension of 35 to 70 mN/m or 60 mN/m or less, optionally 50 mN/m or less, optionally 40 mN/m or less.

16. The amphiphilic cellulose derivative of any one of aspects 1-15, wherein the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative.

17. The amphiphilic cellulose derivative of aspect 16, wherein the nucleation induction time is increased about 2 to about 45 fold.

18. The amphiphilic cellulose derivative of any one of aspects 16-17, wherein the amphiphilic cellulose derivative exhibits a surface tension 50 mN/m or less, preferably 40 mN/m or less, and wherein the nucleation induction time is increased about 2 to 45 fold or 12 to 45 fold.

19. The amphiphilic cellulose derivative of any one of aspects 1-18, wherein the nucleation induction time of the supersaturated aqueous solution is greater than 0 minutes but less than 25 minutes or less than 10 minutes.

20. An amorphous solid dispersion comprising:
an amphiphilic cellulose derivative according to any one of aspects 1-19; and
one or more agents, optionally wherein one or more of the one or more agents is poorly water soluble.

21. An amorphous solid dispersion comprising:
a cellulose or a cellulose derivative comprising one or more α,ω-dicarboxylic acid mono-ester (“ω-carboxyalkanoate ester”) substituents; and
one or more agents, optionally wherein one or more of the one or more agents is poorly water soluble.

22. The amorphous solid dispersion of aspect 21, wherein the one or more agents are crystalline organic molecule(s).

23. The amorphous solid dispersion of any one of aspects 21-22, wherein the one or more agents are each independently selected from a weakly basic pharmaceutical compound, a neutral pharmaceutical compound, or a weakly acidic pharmaceutical compound.

24. The amorphous solid dispersion of any one of aspects 21-23, wherein one or more of the one or more agents is a therapeutic agent.

25. The amorphous solid dispersion of any one of aspects 21-24, wherein one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof.

26. The amorphous solid dispersion of any one of aspects 21-25, wherein the one or more agents are each independently selected from nonsteroidal anti-inflammatory agents (NSAIDs), antifungal agents, chemotherapeutics, or any combination thereof.

27. The amorphous solid dispersion of aspect 26, wherein the one or more agents are each independently selected from Celecoxib, Enzalutamide, Posaconazole, or any combination thereof.

28. The amorphous solid dispersion of any one of claims 21-27, wherein the one or more agents, optionally one or more of the poorly water soluble agents, has increased bioavailability as compared to its free form.

29. The amorphous solid dispersion of any one of aspects 21-28, wherein the one or more agents, optionally one or more of the poorly soluble agents, has increased oral bioavailability as compared to its free form.

30. The amorphous solid dispersion of any one of aspects 21-29, wherein the amorphous solid dispersion is prepared by a method comprising:
reacting a cellulose or cellulose derivative and an aliphatic cyclic anhydride, wherein reacting results in the ω-carboxyalkanoate ester substituent of the amphiphilic cellulose derivative.

31. The amorphous solid dispersion of aspect 30, wherein the aliphatic cyclic anhydride is a succinic anhydride, glutaric anhydride, or a substituted derivative thereof, optionally wherein the derivative thereof comprises an alkanoate substituent, an alkyl substituent, a hydroxyalkyl substituent, or any combination thereof.

32. The amorphous solid dispersion of any one of aspects 30-31, wherein reacting comprises a ring opening reaction.

33. The amorphous solid dispersion of any one of aspects 30-32, wherein the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof.

34. The amorphous solid dispersion of any one of aspects 30-33, wherein the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents.

35. The amorphous solid dispersion of any one of aspects 30-34, wherein the cellulose or cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

36. The amorphous solid dispersion of any one of aspects 30-35, wherein the amphiphilic cellulose derivative does not comprise oligomeric anhydrides, poly(anhydrides), side chains thereof, or any combination thereof, optionally crosslinked or capable of forming crosslinks between hydroxyl groups of different molecules of the amphiphilic cellulose derivative; and/or
wherein the method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both.

37. The amorphous solid dispersion of aspect 36, wherein one or more side reactions are eliminated, reduced, minimized, or any combination thereof by optimizing one or more reaction parameters, optionally wherein the one or more reaction parameters is reaction time, reaction temperature, stoichiometry, solvent, product isolation, product isolation, or any combination thereof.

38. The amorphous solid dispersion of any one of aspects 21-38, wherein the amorphous solid dispersion is prepared by a method comprising:
dissolving the one or more agents, optionally one or more poorly water soluble agents, and one or more amphiphilic cellulose derivatives in one or more organic solvent(s), optionally chosen from an alcohol, tetrahydrofuran, chloroform, acetone, dimethylsulfoxide, dimethylformamide, and dimethylacetamide; and
removing the solvent from one or more agents and the one or more amphiphilic cellulose derivatives, thus forming the amorphous solid dispersion.

39. The solid dispersion of any one of aspects 21-38, wherein the amorphous solid dispersion is prepared by thermal extrusion or electrospinning of one or more amphiphilic cellulose derivatives and one or more agents, optionally one or more therapeutic agents, so as to form the amorphous solid dispersion.

40. The amorphous solid dispersion of any one of aspects 38-39, wherein one or more of the one or more amphiphilic cellulose derivatives comprises one or more ω-carboxyalkanoate ester substituents.

41. The amorphous solid dispersion of any one of aspects 38-40, wherein the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents.

42. The amorphous solid dispersion of any one of aspects 38-41, wherein the amphiphilic cellulose derivative comprises acetate substituents, propionate substituents, butyrate substituents, hydroxyalkyl ether substituents, hydroxy functional polyether substituents, or combinations thereof.

43. The amorphous solid dispersion of any one of aspects 38-42, wherein the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

44. The amorphous solid dispersion of any one of aspects 38-43, wherein the amphiphilic cellulose derivative comprises a number-average molecular weight (Mn) of 15,000 g/mol or greater.

45. The amorphous solid dispersion of any one of aspects 38-44, wherein the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

46. The amorphous solid dispersion of any one of aspects 38-45, wherein the amphiphilic cellulose derivative is water soluble at 50 mg/mL or greater.

47. The amorphous solid dispersion of any one of aspects 38-46, wherein the amphiphilic cellulose derivative exhibits a surface tension of 35 to 70 mN/m.

48. The amorphous solid dispersion of any one of aspects 38-47, wherein the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold.

49. The amorphous solid dispersion of aspect 48, wherein the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

50. The amorphous solid dispersion of any one of aspects 48-49, wherein the nucleation induction time of the supersaturated aqueous solution is greater than 0 minutes but less than 25 minutes or less than 10 minutes.

51. The amorphous solid dispersion of aspect 47, wherein the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold.

52. The amorphous solid dispersion of any one of aspects 49-51, wherein the one or more amphiphilic cellulose derivatives are prepared by a method comprising mono-esterification of a plurality of diacids or derivatives, with a plurality of hydroxyl groups of a cellulose or a cellulose derivative.

53. The amorphous solid dispersion of aspect 52, wherein each of the plurality of diacids are independently dicarboxylic acids, optionally α,ω-dicarboxylic acids, or more optionally C₄ or higher α,ω-dicarboxylic acids.

54. The amorphous solid dispersion of aspect 53, wherein the dicarboxylic acids are each independently selected from succinic acid, glutaric acid any substituted derivative thereof, and any combination thereof, optionally wherein the derivative thereof comprises an alkanoate substituent, an alkyl substituent, a hydroxyalkyl substituent, or any combination thereof.

55. The amorphous solid dispersion of aspect 52, wherein the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides.

56. The amorphous solid dispersion of any one of aspects 38-55, wherein the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

57. A pharmaceutical formulation comprising:
an amorphous solid dispersion of any one of aspects 20-56.

58. The pharmaceutical formulation of aspect 57, wherein the one or more agents, optionally one or more of the poorly soluble agents, has increased oral bioavailability as compared to the free form of the one or more agents.

59. The pharmaceutical formulation of any one of aspects 57-58, wherein the amorphous solid dispersion is in the form of granulated particles or a tablet.

60. A kit comprising an amorphous solid dispersion of any one of aspects 20-56 or a pharmaceutical formulation thereof, optionally a pharmaceutical formulation of any one of aspects 57-59.

61. A method of delivering one or more agents to a subject in need thereof, the method comprising: administering to the subject in need thereof an amorphous solid dispersion of any one of aspects 20-56 or a pharmaceutical formulation thereof, optionally a pharmaceutical formulation of any one of aspects 57-59.

62. The method of aspect 61, wherein the amorphous solid dispersion or pharmaceutical formulation thereof is effective to treat or prevent a disease, condition, disorder, or a symptom thereof in the subject.

63. A method of preparing amphiphilic cellulose derivatives and/or an amorphous solid dispersion thereof comprising one or more α,ω-dicarboxylic acid mono-ester (“ω-carboxyalkanoate ester”) substituents, the method comprising:
reacting a cellulose or cellulose derivative and an aliphatic cyclic anhydride, wherein reacting results in the ω-carboxyalkanoate ester substituents of the amphiphilic cellulose derivative.

64. The method of aspect 63, wherein the aliphatic cyclic anhydride is a succinic anhydride, glutaric anhydride, a substituted derivative thereof, or any combination thereof, optionally wherein the derivative thereof comprises an alkanoate substituent, an alkyl substituent, a hydroxyalkyl substituent, or any combination thereof.

65. The method of any one of aspects 63-64, wherein reacting comprises a ring opening reaction.

66. The method of any one of aspects 63-65, wherein the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof.

67. The method of any one of aspects 63-66, wherein the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents.

68. The method of any one of aspects 63-67, wherein the cellulose or cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

69. The method of any one of aspects 63-68, wherein the method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both.

70. The method of aspect 69, wherein the side reactions are eliminated, reduced, or minimized by optimizing one or more reaction parameters, optionally wherein the one or more reaction parameters is reaction time, reaction temperature, stoichiometry, solvent, product isolation, product isolation, or any combination thereof.

71. A method of preparing an amphiphilic cellulose derivative, the method comprising:
mono-esterification of a plurality of diacids or derivatives with a plurality of hydroxyl groups of a cellulose or a cellulose derivative, optionally wherein the plurality of diacids or derivates thereof comprise anhydrides.

72. The method of aspect 71, wherein the amphiphilic cellulose derivative is capable of inhibiting crystallization of one or more agents, optionally one or more poorly water soluble agents, from aqueous solution.

73. The method of any one of aspects 71-72, wherein the method mono-esterifies an average of at least 50% of the available hydroxyl groups of a cellulose or a cellulose derivative.

74. The method of any one of aspects 71-73, wherein each of the plurality of diacids are independently dicarboxylic acids, optionally α,ω-dicarboxylic acids, or more optionally C₄ or higher α,ω-dicarboxylic acids.

75. The method of aspects 74, wherein the dicarboxylic acids are each independently selected from succinic acid, glutaric acid, a substituted derivative thereof, and any combination thereof, optionally wherein the derivative thereof comprises an alkanoate substituent, an alkyl substituent, a hydroxyalkyl substituent, and any combination thereof.

76. A method of preparing an amphiphilic cellulose derivative, the method comprising: mono-esterification of a plurality of diacids or derivatives with a plurality of hydroxyl groups of a cellulose or a cellulose derivative, optionally wherein the plurality of diacids or derivates thereof comprise anhydrides.

77. The method of any one of aspects 71-75, wherein the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides.

78. The method of aspect 76, wherein the diacids are dicarboxylic acids and are each independently chosen from succinic acid and glutaric acid.

79. The method of any one of aspect 71-78, wherein the cellulose is microcrystalline cellulose.

80. The method of any one of aspect 76-79, wherein the cellulose derivative comprises an average degree of substitution of available hydroxyl groups of 1.8 or greater.

81. The method of any one of aspect 76-80, wherein the cellulose derivative comprises ester substituents having the formula —C(═O)R, ether substituents having the formula —OR, or combinations thereof.

82. The method of any one of aspects 76-81, wherein R is independently chosen from hydroxyl groups, alkyl ester substituents, and alkoxide ester substituents.

83. The method of any one of aspects 76-82, wherein the cellulose derivative comprises one or more acetate substituents, one or more propionate substituents, one or more butyrate substituents, one or more hydroxyalkyl substituents, one or more hydroxy functional polyether substituents, or any combination thereof.

84. The method of any one of aspects 76-83, wherein the cellulose derivative is cellulose acetate, cellulose acetate butyrate, cellulose acetate propionate, hydroxypropyl cellulose, or hydroxyethyl cellulose.

85. The method of any one of aspects 76-84, wherein the amphiphilic cellulose derivative comprises a number-average molecular weight (Mn) of 15,000 g/mol or greater.

86. The method of any one of aspects 76-85, wherein the amphiphilic cellulose derivative is soluble in one or more organic solvents, wherein the one or more organic solvents are optionally chosen from an alcohol, tetrahydrofuran, chloroform, acetone, dimethyl sulfoxide, dimethylformamide, and dimethylacetamide.

87. The method of any one of aspects 71-86, wherein the amphiphilic cellulose derivative is water soluble at 50 mg/mL or greater.

88. The method of any one of aspects 71-87, wherein the amphiphilic cellulose derivative exhibits a surface tension of 35 to 70 mN/m or 60 mN/m or less, preferably less than 50 mN/m.

89. The method of any one of aspects 71-88 wherein the amphiphilic cellulose derivative is capable of increasing nucleation induction time of a supersaturated aqueous solution comprising the amphiphilic cellulose derivative and one or more agents as compared to a solution without the amphiphilic cellulose derivative.

90. The method of aspect 89, wherein the nucleation induction time is increased about 2 to about 45 fold.

91. The method of aspect 90, wherein the amphiphilic cellulose derivative exhibits a surface tension of 50 mN/m or less, optionally less than 40 mN/m, and is capable of increasing nucleation induction time of the supersaturated aqueous solution 2 to 45 fold or 12 to 45 fold.

92. The method of any one of aspects 88-91, wherein the amphiphilic cellulose derivative is water soluble.

93. The method of any one of aspects 88-92, wherein the amphiphilic cellulose derivative comprises an average of at least 25% carboxylic acid functional substituents based on total constituents.

94. The method of any one of aspects 88-93, wherein the amphiphilic cellulose derivative exhibits a surface tension of 60 mN/m or less, preferably less than 50 mN/m.

95. The method of any one of aspects 88-94, wherein the supersaturated aqueous solution comprises a loading ratio of the one or more agents, optionally one or more poorly soluble agents, and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

96. The method of any one of aspects 88-95, wherein the nucleation induction time of the supersaturated aqueous solution is 25 minutes or less or 10 minutes or less.

97. A method of forming an amorphous solid dispersion (ASD) comprising one or more agents, optionally one or more poorly soluble agents, the method comprising the steps of:
dissolving the one or more agents, optionally one or more poorly soluble agents, and one or more amphiphilic cellulose derivatives in one or more organic solvent(s), optionally chosen from an alcohol, tetrahydrofuran, chloroform, acetone, dimethylsulfoxide, dimethylformamide, and dimethylacetamide; and
removing the solvent from one or more agents, optionally one or more poorly soluble agents, and the one or more amphiphilic cellulose derivatives, thus forming the amorphous solid dispersion.

98. A method of forming an amorphous solid dispersion (ASD) comprising:
thermal extrusion of one or more amphiphilic cellulose derivatives and one or more agents, optionally one or more therapeutic agents, so as to form the amorphous solid dispersion.

99. The method of forming the amorphous solid dispersion of any one of aspects 97-98, wherein one or more of the one or more amphiphilic cellulose derivatives comprises one or more ω-carboxyalkanoate ester substituents.

100. The method of forming the amorphous solid dispersion of any one of aspects 97-99, wherein the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents.

101. The method of forming the amorphous solid dispersion of any one of aspects 97-100, wherein the amphiphilic cellulose derivative comprises acetate substituents, propionate substituents, butyrate substituents, hydroxy functional polyether substituents, or combinations thereof.

102. The method of forming the amorphous solid dispersion of any one of aspects 97-101, wherein the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

103. The method of forming the amorphous solid dispersion of any one of aspects 97-102, wherein the amphiphilic cellulose derivative comprises a number-average molecular weight (Mn) of 15,000 g/mol or greater.

104. The method of forming the amorphous solid dispersion of any one of aspects 97-103, wherein the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide.

105. The method of forming the amorphous solid dispersion of any one of aspects 97-104, wherein the amphiphilic cellulose derivative is water soluble at 50 mg/mL or greater.

106. The method of forming the amorphous solid dispersion of any one of aspects 97-105, wherein the amphiphilic cellulose derivative exhibits a surface tension of 35 to 70 mN/m or 60 mN/m or less.

107. The method of forming an amorphous solid dispersion of any one of aspects 97-106, wherein the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold.

108. The method of forming an amorphous solid dispersion of aspect 107, wherein the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

109. The method of forming an amorphous solid dispersion of any one of aspects 107 or 108, wherein the nucleation induction time of the supersaturated aqueous solution is greater than 0 minutes but less than 25 minutes or less than 10 minutes.

110. The method of forming an amorphous solid dispersion of any one of aspects 107-109, wherein the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold.

111. The method of forming an amorphous solid dispersion of any one of aspects 97-110 further comprising grinding the amorphous solid dispersion to a desired particle size, and optionally compressing the amorphous solid dispersion particles into tablets.

112. The method of forming an amorphous solid dispersion of any one of aspects 97-111, wherein the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio.

113. The method of any one of aspects 97-112 wherein the one or more agents is/are crystalline organic molecule(s).

114. The method of any one of aspects 97-113 wherein the one or more agents are each independently selected from a weakly basic pharmaceutical compound, a neutral pharmaceutical compound, or a weakly acidic pharmaceutical compound.

115. The method of any one of aspects 97-114, wherein one or more of the one or more agents is a therapeutic agent.

116. The method of any one of aspects 97-115, wherein one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof.

117. The method of any one of aspects 97-116, wherein the one or more agents are each independently selected from nonsteroidal anti-inflammatory agents (NSAIDs), antifungal agents, chemotherapeutics, or any combination thereof.

118. The method of aspect 117, wherein the one or more agents are each independently selected from Celecoxib, Enzalutamide, Posaconazole, or any combination thereof.

119. An amphiphilic cellulose derivative comprising one or more ω-carboxyalkanoate ester substituents, wherein the amphiphilic cellulose derivative is made by a method as in any one of aspects 70-95.

120. The amphiphilic cellulose derivative of aspect 119, wherein the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

121. An amphiphilic cellulose derivative prepared by the method of any one of aspect 63 to 120.

122. The amphiphilic cellulose derivative of aspect 121, wherein the amphiphilic cellulose derivative comprises one or more ω-carboxyalkanoate ester substituents.

1. An amphiphilic cellulose derivative comprising:
a cellulose or a cellulose derivative, wherein one or more hydroxyl groups of the cellulose or the cellulose derivative are replaced by α,ω-dicarboxylic acid mono-ester (“ω-carboxyalkanoate ester”) substituents, optionally C₄ or higher ω-carboxyalkanoate ester substituents, any substituted derivatives thereof, or any combination thereof, and wherein the cellulose or the cellulose derivative is not crosslinked.

2. The amphiphilic cellulose derivative of claim 1, wherein the amphiphilic cellulose derivative
(a) does not comprise oligomeric anhydrides, poly(anhydrides), side chains thereof, or any combination thereof, optionally crosslinked or capable of forming crosslinks between hydroxyl groups of different molecules of the amphiphilic cellulose derivative;
(b) comprises a 1/1 or greater molar ratio of ω-carboxyalkanoate ester substituents to available hydroxyl groups, and/or wherein the amphiphilic cellulose derivative comprises an average of 25% or greater ω-carboxyalkanoate ester substituents based on total substituents;
(c) is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate;
(d) the amphiphilic cellulose derivative comprises a number-average molecular weight (Mn) of 15,000 g/mol or greater;
(e) is soluble in an organic solvent, optionally chosen from an alcohol, tetrahydrofuran, chloroform, acetone, dimethyl sulfoxide, dimethylformamide, dimethylacetamide, and any combination thereof;
(f) is water soluble at 50 mg/mL or greater;
(g) exhibits a surface tension of 35 to 70 mN/m or 60 mN/m or less, optionally 50 mN/m or less, optionally 40 mN/m or less;
(h) is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold, and optionally wherein the nucleation induction time of the supersaturated aqueous solution is greater than 0 minutes but less than 25 minutes or less than 10 minutes;
(i) exhibits a surface tension 50 mN/m or less, preferably 40 mN/m or less, and wherein the nucleation induction time is increased about 2 to 45 fold or 12 to 45 fold; or (d) any combination thereof.

3. The amphiphilic cellulose derivative of claim 1, wherein
(a) the ω-carboxyalkanoate ester substituents comprise a terminal carboxylic acid group or a monovalent salt thereof, optionally an alkali metal or a quaternary ammonium salt thereof;
(b) the ω-carboxyalkanoate ester substituents are linear C₄ or higher ω-carboxyalkanoate ester substituents chosen from succinate mono-ester substituents, glutarate mono-ester substituents, any substituted derivatives thereof, and any combination thereof, optionally wherein the substituted derivatives thereof comprise alkanoate substituents, alkyl substituents, hydroxyalkyl substituents, or any combination thereof.

4. (canceled)

5. (canceled)

6. The amphiphilic cellulose derivative of claim 1, wherein
(a) the cellulose derivative comprises an average degree of substitution of available hydroxyl groups of 1.8 or greater;
(b) the cellulose derivative comprises ester substituents having the formula —C(═O)R, ether substituents having the formula —OR, or any combination thereof, optionally wherein R is independently chosen at each occurrence from hydroxyl groups, alkyl ester substituents, alkoxide ester substituents, and any combination thereof, or optionally wherein R is independently chosen at each occurrence from —OH or an alkyl;
(c) the cellulose derivative comprises acetate substituents, propionate substituents, butyrate substituents, hydroxyalkyl substituents, hydroxy functional polyether substituents, or any combination thereof;
(d) the cellulose derivative is cellulose acetate, cellulose propionate, cellulose acetate propionate, cellulose acetate butyrate, hydroxypropyl cellulose, hydroxyethyl cellulose, or any combination thereof;
(e) the cellulose or cellulose derivative is microcrystalline cellulose (MCC), cellulose acetate 320S (CA320S), cellulose acetate 398 (CA398), cellulose acetate butyrate (CAB), CAB-553-0.4, cellulose acetate propionate (CAP), CAP-504.02, hydroxypropyl cellulose, hydroxyethyl cellulose, or any combination thereof; or
(f) any combination thereof.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. An amorphous solid dispersion comprising:
an amphiphilic cellulose derivative according to claim 1 or a cellulose or a cellulose derivative comprising one or more α,ω-dicarboxylic acid mono-ester (“ω-carboxyalkanoate ester”) substituents; and
one or more agents, optionally wherein
(a) one or more of the one or more agents is poorly water soluble;
(b) the one or more agents are crystalline organic molecule(s);
(c) the one or more agents are each independently selected from a weakly basic pharmaceutical compound, a neutral pharmaceutical compound, or a weakly acidic pharmaceutical compound;
(d) one or more of the one or more agents is a therapeutic agent;
(e) one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof;
(f) the one or more agents are each independently selected from nonsteroidal anti-inflammatory agents (NSAIDs), antifungal agents, chemotherapeutics, or any combination thereof;
(g) the one or more agents are each independently selected from Celecoxib, Enzalutamide, Posaconazole, or any combination thereof;
(h) the one or more agents, optionally one or more of the poorly water soluble agents, has increased bioavailability as compared to its free form;
(i) the one or more agents, optionally one or more of the poorly soluble agents, has increased oral bioavailability as compared to its free form; or
(j) any combination thereof.

21. (canceled)

22. (canceled)

23. (canceled)

24. (canceled)

25. (canceled)

26. (canceled)

27. (canceled)

28. (canceled)

29. (canceled)

30. The amorphous solid dispersion of claim 21, wherein the amorphous solid dispersion is prepared by a method comprising:
(i) reacting a cellulose or cellulose derivative and an aliphatic cyclic anhydride, wherein reacting results in the ω-carboxyalkanoate ester substituent of the amphiphilic cellulose derivative;
(ii) dissolving the one or more agents, optionally one or more poorly water soluble agents, and one or more amphiphilic cellulose derivatives in one or more organic solvent(s), optionally chosen from an alcohol, tetrahydrofuran, chloroform, acetone, dimethylsulfoxide, dimethylformamide, and dimethylacetamide; and removing the solvent from one or more agents and the one or more amphiphilic cellulose derivatives, thus forming the amorphous solid dispersion; or
(iii) thermal extrusion or electrospinning of one or more amphiphilic cellulose derivatives and one or more agents, optionally one or more therapeutic agents, so as to form the amorphous solid dispersion.

31. The amorphous solid dispersion of claim 30, wherein the amorphous solid dispersion is prepared by (i) and wherein
(a) the aliphatic cyclic anhydride is a succinic anhydride, glutaric anhydride, or a substituted derivative thereof, optionally wherein the derivative thereof comprises an alkanoate substituent, an alkyl substituent, a hydroxyalkyl substituent, or any combination thereof;
(b) reacting comprises a ring opening reaction;
(c) the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof;
(d) the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents;
(e) the cellulose or cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof;
(f) the amphiphilic cellulose derivative does not comprise oligomeric anhydrides, poly(anhydrides), side chains thereof, or any combination thereof, optionally crosslinked or capable of forming crosslinks between hydroxyl groups of different molecules of the amphiphilic cellulose derivative;
(g) method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both; optionally wherein one or more side reactions are eliminated, reduced, minimized, or any combination thereof by optimizing one or more reaction parameters, optionally wherein the one or more reaction parameters is reaction time, reaction temperature, stoichiometry, solvent, product isolation, product isolation, or any combination thereof.

32. (canceled)

33. (canceled)

34. (canceled)

35. (canceled)

36. (canceled)

37. (canceled)

38. (canceled)

39. (canceled)

40. The amorphous solid dispersion of claim 30, wherein the amorphous solid dispersion is prepared by (ii) and wherein
(a) one or more of the one or more amphiphilic cellulose derivatives comprises one or more ω-carboxyalkanoate ester substituents;
(b) the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents;
(c) the amphiphilic cellulose derivative comprises acetate substituents, propionate substituents, butyrate substituents, hydroxyalkyl ether substituents, hydroxy functional polyether substituents, or combinations thereof;
(d) the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof;
(e) the amphiphilic cellulose derivative comprises a number-average molecular weight (Mn) of 15,000 g/mol or greater;
(f) the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide;
(g) the amphiphilic cellulose derivative is water soluble at 50 mg/mL or greater;
(h) the amphiphilic cellulose derivative exhibits a surface tension of 35 to 70 mN/m or 60 mN/m or less;
(i) the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold, optionally wherein the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio, and optionally wherein the nucleation induction time of the supersaturated aqueous solution is greater than 0 minutes but less than 25 minutes or less than 10 minutes;
(j) the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold;
(k) the one or more amphiphilic cellulose derivatives are prepared by a method comprising mono-esterification of a plurality of diacids or derivatives, with a plurality of hydroxyl groups of a cellulose or a cellulose derivative, optionally wherein each of the plurality of diacids are independently dicarboxylic acids, optionally α,ω-dicarboxylic acids, or more optionally C4 or higher α,ω-dicarboxylic acids, optionally wherein the dicarboxylic acids are each independently selected from succinic acid, glutaric acid any substituted derivative thereof, and any combination thereof, optionally wherein the derivative thereof comprises an alkanoate substituent, an alkyl substituent, a hydroxyalkyl substituent, or any combination thereof, optionally wherein the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides;
(l) the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio; or
(m) any combination thereof.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A pharmaceutical formulation comprising:
an amorphous solid dispersion of claim 20, optionally wherein the one or more agents, optionally one or more of the poorly soluble agents, has increased oral bioavailability as compared to the free form of the one or more agents, and optionally wherein the amorphous solid dispersion is in the form of granulated particles or a tablet.

58. (canceled)

59. (canceled)

60. A kit comprising an amorphous solid dispersion of claim 20 or a pharmaceutical formulation thereof, optionally wherein the one or more agents, optionally one or more of the poorly soluble agents, has increased oral bioavailability as compared to the free form of the one or more agents, optionally wherein the amorphous solid dispersion is in the form of granulated particles or a tablet.

61. A method of delivering one or more agents to a subject in need thereof, the method comprising:
administering to the subject in need thereof an amorphous solid dispersion of claim 20 or a pharmaceutical formulation thereof, optionally wherein the one or more agents, optionally one or more of the poorly soluble agents, has increased oral bioavailability as compared to the free form of the one or more agents, optionally wherein the amorphous solid dispersion is in the form of granulated particles or a tablet, and optionally wherein the amorphous solid dispersion or pharmaceutical formulation thereof is effective to treat or prevent a disease, condition, disorder, or a symptom thereof in the subject.

62. (canceled)

63. A method of preparing amphiphilic cellulose derivatives and/or an amorphous solid dispersion thereof comprising one or more α,ω-dicarboxylic acid mono-ester (“ω-carboxyalkanoate ester”) substituents, the method comprising:
reacting a cellulose or cellulose derivative and an aliphatic cyclic anhydride, wherein reacting results in the ω-carboxyalkanoate ester substituents of the amphiphilic cellulose derivative, optionally wherein reacting comprises a ring opening reaction; or
mono-esterification of a plurality of diacids or derivatives with a plurality of hydroxyl groups of a cellulose or a cellulose derivative, optionally wherein the plurality of diacids or derivates thereof comprise anhydrides.

64. The method of claim 63, wherein
(a)_the aliphatic cyclic anhydride is a succinic anhydride, glutaric anhydride, a substituted derivative thereof, or any combination thereof, optionally wherein the derivative thereof comprises an alkanoate substituent, an alkyl substituent, a hydroxyalkyl substituent, or any combination thereof;
(b) the cellulose derivative is a cellulose alkanoate, an alkyl cellulose ether, a hydroxyalkyl cellulose ether, a cellulose acetate propionate, a cellulose acetate butyrate, a cellulose acetate, or any combination thereof;
(c) the cellulose derivative comprises ester substituents having the formula —C(═O)R ether substituents having the formula —OR, or combinations thereof, optionally wherein R is independently chosen from hydroxyl groups, alkyl ester substituents, and alkoxide ester substituents;
(d) the cellulose or cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof;
(e) the method of preparing the amphiphilic cellulose derivative eliminates, reduces, or minimizes one or more side reactions, optionally wherein the one or more side reactions are homopolymerization of the cyclic anhydride, chain extension of the originally formed ω-carboxyalkanoate to an oligomeric poly(anhydride) side chain of cellulose capable of forming crosslinks via reaction with hydroxyl groups on other cellulose derivative molecules, or both; optionally wherein the side reactions are eliminated, reduced, or minimized by optimizing one or more reaction parameters, optionally wherein the one or more reaction parameters is reaction time, reaction temperature, stoichiometry, solvent, product isolation, product isolation, or any combination thereof; or
(f) any combination thereof.

65. (canceled)

66. (canceled)

67. (canceled)

68. (canceled)

69. (canceled)

70. (canceled)

71. (canceled)

72. The method of claim 63, wherein
(a) the amphiphilic cellulose derivative is capable of inhibiting crystallization of one or more agents, optionally one or more poorly water soluble agents, from aqueous solution;
(b) the method mono-esterifies an average of at least 50% of the available hydroxyl groups of a cellulose or a cellulose derivative;
(c) each of the plurality of diacids are independently dicarboxylic acids, optionally α,ω-dicarboxylic acids, or more optionally C₄ or higher α,ω-dicarboxylic acids, optionally wherein the dicarboxylic acids are each independently selected from succinic acid, glutaric acid, a substituted derivative thereof, and any combination thereof, optionally wherein the derivative thereof comprises an alkanoate substituent, an alkyl substituent, a hydroxyalkyl substituent, and any combination thereof;
(d) the plurality of diacid derivatives are anhydrides, optionally wherein the anhydrides are independently carboxylic anhydrides, optionally linear or cyclic anhydrides, optionally C₄-C₈ or higher linear or cyclic anhydrides, optionally C₄ or C₅ linear or cyclic anhydrides;
(e) the diacids are dicarboxylic acids and are each independently chosen from succinic acid and glutaric acid;
(f) the cellulose is microcrystalline cellulose;
(g) the cellulose derivative comprises an average degree of substitution of available hydroxyl groups of 1.8 or greater;
(h) the cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents;
(i) the cellulose derivative comprises one or more acetate substituents, one or more propionate substituents, one or more butyrate substituents, one or more hydroxyalkyl substituents, one or more hydroxy functional polyether substituents, or any combination thereof;
(j) the cellulose derivative is cellulose acetate, cellulose acetate butyrate, cellulose acetate propionate, hydroxypropyl cellulose, or hydroxyethyl cellulose;
(k) the amphiphilic cellulose derivative comprises a number-average molecular weight (Mn) of 15,000 g/mol or greater;
(l) the amphiphilic cellulose derivative is soluble in one or more organic solvents, wherein the one or more organic solvents are optionally chosen from an alcohol, tetrahydrofuran, chloroform, acetone, dimethyl sulfoxide, dimethylformamide, and dimethylacetamide;
(m) the amphiphilic cellulose derivative is water soluble at 50 mg/mL or greater;
(n) the amphiphilic cellulose derivative exhibits a surface tension of 35 to 70 mN/m or 60 mN/m or less, or less than 50 mN/m;
(o) the amphiphilic cellulose derivative is capable of increasing nucleation induction time of a supersaturated aqueous solution comprising the amphiphilic cellulose derivative and one or more agents as compared to a solution without the amphiphilic cellulose derivative, optionally the nucleation induction time is increased about 2 to about 45 fold;
(p) the amphiphilic cellulose derivative exhibits a surface tension of 50 mN/m or less, optionally less than 40 mN/m, and is capable of increasing nucleation induction time of the supersaturated aqueous solution 2 to 45 fold or 12 to 45 fold; optionally wherein the supersaturated aqueous solution comprises a loading ratio of the one or more agents, optionally one or more poorly soluble agents, and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio; and optionally wherein the nucleation induction time of the supersaturated aqueous solution is 25 minutes or less or 10 minutes or less;
(q) the amphiphilic cellulose derivative is water soluble;
(r) the amphiphilic cellulose derivative comprises an average of at least 25% carboxylic acid functional substituents based on total constituents; or
(s) any combination thereof.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. A method of forming an amorphous solid dispersion (ASD) comprising one or more agents, optionally one or more poorly soluble agents, the method comprising the steps of:
(i) dissolving the one or more agents, optionally one or more poorly soluble agents, and one or more amphiphilic cellulose derivatives in one or more organic solvent(s), optionally chosen from an alcohol, tetrahydrofuran, chloroform, acetone, dimethylsulfoxide, dimethylformamide, and dimethylacetamide; and removing the solvent from one or more agents, optionally one or more poorly soluble agents, and the one or more amphiphilic cellulose derivatives, thus forming the amorphous solid dispersion; or
(ii) thermal extrusion of one or more amphiphilic cellulose derivatives and one or more agents, optionally one or more therapeutic agents, so as to form the amorphous solid dispersion.

98. (canceled)

99. The method of forming the amorphous solid dispersion of claim 97, wherein
(a) one or more of the one or more amphiphilic cellulose derivatives comprises one or more ω-carboxyalkanoate ester substituents;
(b) the amphiphilic cellulose derivative comprises ester substituents having the formula —C(═O)R, ether substituents having the formula —OR, or combinations thereof, optionally wherein R is independently chosen from hydroxyl groups, alkyl ester substituents, and alkoxide ester substituents;
(c) the amphiphilic cellulose derivative comprises acetate substituents, propionate substituents, butyrate substituents, hydroxy functional polyether substituents, or combinations thereof;
(d) the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof;
(e) the amphiphilic cellulose derivative comprises a number-average molecular weight (Mn) of 15,000 g/mol or greater;
(f) the amphiphilic cellulose derivative is soluble in one or more organic solvent(s), optionally chosen from an alcohol, tetrahydrofuran, chloroform, acetone, dimethyl sulfoxide, dimethylformamide, or dimethylacetamide;
(g) the amphiphilic cellulose derivative is water soluble at 50 mg/mL or greater;
(h) the amphiphilic cellulose derivative exhibits a surface tension of 35 to 70 mN/m or 60 mN/m or less;
(i) the amphiphilic cellulose derivative is capable of increasing nucleation induction time of a supersaturated aqueous solution comprising the amphiphilic cellulose derivative and one or more agents, optionally one or more poorly soluble agents, as compared to a solution without the amphiphilic cellulose derivative, optionally wherein the nucleation induction time is increased about 2 to about 45 fold,
optionally wherein the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio,
optionally wherein the supersaturated aqueous solution comprises a loading ratio of the one or more agents and the amphiphilic cellulose derivative of 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio; and
optionally wherein the nucleation induction time of the supersaturated aqueous solution is greater than 0 minutes but less than 25 minutes or less than 10 minutes;
(j) optionally wherein the amphiphilic cellulose derivative exhibits a surface tension of 50 mN/m or less, preferably less than 40 mN/m and is capable of increasing nucleation induction time of the supersaturated aqueous solution 2 to 45 fold or 12 to 45 fold;
(k) the one or more agents and the one or more amphiphilic cellulose derivatives are loaded in a 1:99 to 50:50 wt./wt. ratio, optionally a 1:99 wt./wt. ratio, a 5:95 wt./wt. ratio, a 10:90 wt./wt. ratio, a 20:80 wt./wt. ratio, a 30:40 wt./wt. ratio, a 40:60 wt./wt. ratio, or a 50:50 wt./wt. ratio;
(l) the one or more agents is/are crystalline organic molecule(s);
(m) the one or more agents are each independently selected from a weakly basic pharmaceutical compound, a neutral pharmaceutical compound, or a weakly acidic pharmaceutical compound;
(n) one or more of the one or more agents is a therapeutic agent;
(o) one or more of the one or more agents is an immunomodulator, an antipyretic, an anxiolytic, an antipsychotic, an anticonvulsant, an analgesic, an antispasmodic, an anti-inflammatory, an antihistamine, an anti-infective, a chemotherapeutic, a vasomodulator, an anti-diabetic, a radiation sensitizer, a chemotherapeutic sensitizer, an antiviral, an antifungal, an antibacterial, an immunosuppressant, or any combination thereof;
(p) the one or more agents are each independently selected from nonsteroidal anti-inflammatory agents (NSAIDs), antifungal agents, chemotherapeutics, or any combination thereof;
(q) the one or more agents are each independently selected from Celecoxib, Enzalutamide, Posaconazole, or any combination thereof; or
(r) any combination thereof.

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. The method of forming an amorphous solid dispersion of claim 97, further comprising grinding the amorphous solid dispersion to a desired particle size, and optionally compressing the amorphous solid dispersion particles into tablets.

112. (canceled)

113. (canceled)

114. (canceled)

115. (canceled)

116. (canceled)

117. (canceled)

118. (canceled)

119. An amphiphilic cellulose derivative comprising one or more ω-carboxyalkanoate ester substituents, wherein the amphiphilic cellulose derivative is made by a method as in claim 63, optionally wherein the amphiphilic cellulose derivative is microcrystalline cellulose (MCC) succinate, cellulose acetate 320S (CA320S) succinate (low degree of substitution), cellulose acetate 320S (CA320S) succinate (high degree of substitution), cellulose acetate 320S (CA320S) glutarate, cellulose acetate CA398 (CA398) succinate, cellulose acetate butyrate (CAB-553-0.4) succinate, or cellulose acetate propionate (CAP-504-0.2) glutarate, or cellulose acetate glutarate succinate, or any combination thereof.

120. (canceled)

121. An amphiphilic cellulose derivative prepared by the method of claim 63, optionally wherein the amphiphilic cellulose derivative comprises one or more ω-carboxyalkanoate ester substituents.

122. (canceled)
